(19) United States
(12) Patent Application Publication
(10) Pub. No.: US 2026/0083759 A1
(43) Pub. Date: Mar. 26, 2026

(54) Combinations Comprising Psychedelics for the Treatment of Schizophrenia and Other Neuropsychiatric and Neurologic Disorders

(71) Applicants: HADASIT MEDICAL RESEARCH SERVICES AND DEVELOPMENT LTD, Jerusalem (IL); EZRATH NASHIM - THE RABANIT HERZOG MEMORIAL HOSPITAL, Jerusalem (IL)

(72) Inventors: Bernard LERER, Alon Shvut (IL); Uriel HERESCO, Jerusalem (IL)

(21) Appl. No.: 19/109,313

(22) PCT Filed: Aug. 29, 2023
(86) PCT No.: PCT/IL2023/050919

§ 371 Date: Mar. 6, 2025

Related U.S. Application Data
(60) Provisional application No. 63/403,888, filed on Sep. 6, 2022.

(51) Int. Cl. A61K 31/675; A61K 31/137; A61K 31/198; A61K 31/36; A61K 31/382; A61K 31/4045; A61K 31/42; A61K 31/4535; A61K 31/454; A61K 31/48; A61K 31/496; A61K 31/4985; A61K 31/519; A61K 31/5415; A61K 31/5513; A61K 31/553; A61K 31/554; A61P 25/18

(52) U.S. Cl. CPC A61K 31/675; A61K 31/137; A61K 31/198; A61K 31/36; A61K 31/382; A61K 31/4045; A61K 31/42; A61K 31/4535; A61K 31/454; A61K 31/48; A61K 31/496; A61K 31/4985; A61K 31/519; A61K 31/5415; A61K 31/5513; A61K 31/553; A61K 31/554; A61P 25/18

(57) ABSTRACT

This invention provides in some aspects, combinations, compositions and kits comprising a psychedelic drug and an NMD A receptor agonist and applications and uses of same for treating, reducing the incidence of, reducing the severity of or improving a pathology of a subject with a neurologic disease or disorder, a neurocognitive disease or disorder, or a motor disease or disorder, or for enhancing neuroplasticity in a subject with a neurologic disease or disorder, a neurocognitive disease or disorder, or a motor disease or disorder making use of the combinations, compositions and kits of this invention.

### BACKGROUND OF THE INVENTION

Schizophrenia is a severe psychiatric disorder that almost 24 million people suffer from and it is the leading cause of long-term psychiatric hospitalization worldwide.

Schizophrenia is marked by the appearance of symptomatology, which includes ‘positive’ symptoms and ‘negative’ symptoms. Positive symptoms of schizophrenia may include delusions, hallucinations, disorganized speech, and behaviour; while negative symptoms may include diminished emotional expression, lack of motivation, social withdrawal, depression, impaired cognitive function and poor inter-personal and occupational functioning. Patients who suffer from chronic schizophrenia are heavily dependent on social services.

Antipsychotic drugs are effective in the acute phase of the disorder but treatment options for chronic schizophrenia are very limited.

Cortical atrophy and neuropil loss are widely reported in patients with chronic schizophrenia, particularly those with prominent negative symptoms.

5-hydroxytryptamine (5-HT), or serotonin is a neurotransmitter that modulates most human behavioral processes. Drugs that target serotonin receptors are widely used in psychiatry and neurology. A number of serotonergic compounds are known, which are hallucinogens (psychedelics). While some of these compounds show promise in treating neuropsychiatric diseases, their hallucinogenic effects limit their use especially in populations susceptible to psychosis.

There remains a need for more effective therapies for treating neuropsychiatric diseases and schizophrenia in particular.

### SUMMARY OF THE INVENTION

This invention relates, inter alia, to novel treatment approaches for treating schizophrenia patients. In some aspects, this invention relates, inter alia, to novel treatment approaches for chronic schizophrenia patients with negative symptoms.

In some aspects of the invention, concurrent treatment agents modulating 5-HT2A receptor function can induce psychological effects in the short term with neuroplastic changes in key brain areas in the longer term, specifically and exceptionally through the combinations described herein.

Psychedelic drugs induce characteristic changes in mood, perception and cognition. Classical tryptaminergic psychedelics bind with high affinity to 5-HT2A receptors. Psychedelic drugs may contribute to neuroplasticity, where enhanced synaptic plasticity has been demonstrated in the form of increases in dendritic spines and enhanced neurogenesis. The potential for psychedelic drugs effects on neuroplasticity would make them highly attractive candidates for the treatment of chronic schizophrenia however their effects on mood, perception and cognition (termed the psychedelic trip and also referred to as hallucinogenic effects) are highly problematic and therefore use of same at present is contraindicated.

Certain antipsychotic drugs improve negative symptoms and cognitive function in patients with chronic schizophrenia, for example, clozapine, may impact same, which was presumed to be through their blockade 5-HT2A receptors.

For the first time herein it is demonstrated herein that combinations of a psychedelic, such as psilocybin (PSIL) and an antipsychotic that possesses 5-HT2A antagonist properties, such as clozapine (CLZ), prevents the induction of positive symptoms of schizophrenia, in animal models approximating the disease.

Furthermore, D-serine acts as a co-agonist at the glycine modulatory site of N-methyl-D-aspartate type (NMDA) glutamate receptors (NMDAR), which plays a key role in synaptic adaptation processes and same are hypofunctional in schizophrenia. D-serine may elicit clinical improvements in negative symptoms and overall symptomatology in patients with chronic schizophrenia, but so far it has not been envisioned that in combination with a psychedelic, these improvements can be far superior than any treatment to date.

In some embodiments, the combination of a psychedelic drug (such as but not limited to psilocybin) and clozapine for the treatment of schizophrenia prevents psychedelic effects of the psychedelic drug and the Examples herein support this.

Without being bound by theory, in some aspects, the ability of the combination to prevent the psychedelic effects of the psychedelic drug is due to the action of clozapine to block 5-HT2A receptors while at the same time allowing the neuroplastic effect of the psychedelic that would improve negative symptoms of schizophrenia. In some aspects, the invention specifically provides for combination therapies that further enhance neuroplasticity in the brain. For example, and representing certain embodiments, NMDA receptor agonists in the combinations of this invention, may promote neuroplasticity, which may be further promoted in their combination with a psychedelic agent as herein described.

In some aspects, the invention specifically contemplates use of agents that modulate the function of NMDA receptors in the cortex and other brain areas in combination with psychedelics and, optionally further including agents modulating 5-HT2A receptor function. Concurrent administration of the two agents (the agents that modulate the function of NMDA receptors in combination with psychedelics) in a single composition is specifically envisioned.

In some aspects of the invention, it is specifically contemplated to use antipsychotic agents possessing 5-HT2A antagonist properties and psychedelics for their effect on treating schizophrenia. Concurrent administration of these two agents in a single composition is specifically envisioned.

In one embodiment, this invention provides a combination therapy comprising a psychedelic drug and an NMDA receptor agonist.

In some embodiments, this invention provides a combination therapy comprising a psychedelic drug and an antipsychotic possessing 5-HT2A antagonist properties and optionally an NMDA receptor agonist or partial agonist.

In other aspects, the combination regimens/treatments of this invention making use of a psychedelic drug (such as but not limited to psilocybin) and NMDAR agonists/partial agonists, (such as, but not limited to D-serine) for the treatment of schizophrenia provides another unique and highly advantageous combination therapy.

Without being bound by theory, in some aspects, the action of the NMDAR agonists/partial agonist, such as D-serine, to enhance NMDA receptor signaling is combined with the activity of the psychedelic drugs (such as but not limited to psilocybin) in enhancing neuroplasticity. The combination of these two helpful protective effects provides, in some aspects, an improvement superior than any treatment to date.

In another aspect of this invention, there are provided combination regimens/treatments making use of a psychedelic drug (such as but not limited to psilocybin) and NMDAR agonists/partial agonists, (such as, but not limited to D-serine) for the treatment of depression, and in some embodiments, particularly in treatment-resistant depression.

Without being bound by theory, in some aspects, D-serine may be effective in treating depression, specifically treatment-resistant depression, whose action may in fact be synergistic when combined with psychedelics, also in some instances reported to be effective in treating depression, specifically treatment-resistant depression. In some aspects of the invention, the concurrent treatment may result in potentiation of NMDA receptor signaling along with enhanced neuroplasticity that could have significant antidepressant effects.

Without being bound by theory, in some aspects, another embodied indication for an NMDAR agonist/partial agonist (such as, D-serine) combination with a psychedelic agent is use in treatment/management/amelioration of symptomatology of posttraumatic stress disorder (PTSD), and in some embodiments, specifically when same is non-responsive to therapy.

According to these aspects, and in some embodiments, the combination regimens/compositions/methods of this invention are particularly useful in treating symptoms of social withdrawal and amotivation, which in some aspects, is associated with negative symptoms in schizophrenia, as well as in subjects suffering from PTSD, and in some embodiments, both groups of subject may also have a high level of depression comorbidity, which in other aspects is therefore suitable for use with the combination regimens/compositions/methods of this invention.

In other embodiments, the combination regimens/compositions/methods of this invention make use of the NMDAR partial agonist D-cycloserine. According to this aspect and in some embodiments, D-cycloserine may be combined with a psychedelic drug (such as but not limited to psilocybin) and may be particularly useful in the treatment of chronic schizophrenia with prominent negative symptoms, and in some embodiments, for use in treatment-resistant depression and PTSD.

In one embodiment, this invention provides a method of treating, reducing the incidence of, reducing the severity of or improving a pathology of a subject with schizophrenia, the method comprising administering to said subject a combination of a psychedelic drug and an antipsychotic possessing 5-HT2A antagonist properties.

According to this aspect, and in some embodiments, treating, reducing the incidence of, reducing the severity of or improving a pathology of a subject with schizophrenia comprises reducing or abrogating negative symptoms of schizophrenia.

In another embodiment, this invention provides a method of treating, reducing the incidence of, reducing the severity of or improving a pathology of a subject with schizophrenia, the method comprising administering to said subject a combination of a psychedelic drug and an NMDA receptor agonist.

In another embodiment, this invention provides a method of treating, reducing the incidence of, reducing the severity of or improving a pathology of a subject with depression, the method comprising administering to said subject a combination of a psychedelic drug and an NMDA receptor agonist.

In another embodiment, this invention provides a method of treating, reducing the incidence of, reducing the severity of or improving a pathology of a subject with post-traumatic stress disorder, the method comprising administering to said subject a combination of a psychedelic drug and an NMDA receptor agonist.

In another embodiment, this invention provides a method of enhancing neuroplasticity in a subject with schizophrenia, depression or post-traumatic stress disorder, the method comprising administering to said subject a combination of a psychedelic drug and an NMDA receptor agonist.

In other embodiments, this invention specifically contemplates methods of treating, reducing the incidence of, reducing the severity of or improving a pathology of a subject with a neuropsychiatric disease or disorder, neurologic disease or disorder, a neurocognitive disease or disorder, or a motor disease or disorder, comprising administering to said subject a combination therapy of this invention.

In some aspects, this invention specifically contemplates a combination therapy of this invention for use in the treating, reducing the incidence of, reducing the severity of or improving a pathology of a subject with a neuropsychiatric disease or disorder, neurologic disease or disorder, a neurocognitive disease or disorder, or a motor disease or disorder.

### BRIEF DESCRIPTION OF THE DRAWINGS

Various embodiments of the combination therapy, compositions and methods of this invention are described herein with reference to the figures wherein:

FIG. 1A-1B plots head twitch responses (HTR) in total and over time (mean+standard error).

FIG. 2A-2E plots HTR in total and over time, with the indicated treatment groups.

FIG. 3A-3E plots the distance travelled, distance moved and center durations in open field tests, in each of the indicated treatment groups.

FIG. 4 plots the effects of PSIL, DSER and their combination on the expression of the GAP43 synaptic protein in different brain regions, 7 days after administration.

FIG. 5 plots the effects of PSIL, DSER and their combination on the expression of the Synaptophysin and SV2A synaptic proteins in different brain regions, 7 days after administration.

FIG. 6 plots the comparative effects of PSIL, DSER and their combination on the expression of the GAP43 and Synaptophysin synaptic proteins in four brain regions, frontal cortex, hippocampus, amygdala and striatum, 7 days after administration.

FIG. 7 schematically depicts an envisioned treatment regimen comprising administration of a tryptaminergic psychedelic drug, such as, but not limited to, psilocybin to patients receiving treatment with clozapine for at least 2 weeks. The same dose of psilocybin is administered once weekly for 4 weeks concurrently with clozapine treatment.

FIG. 8 schematically depicts another envisioned treatment regimen comprising administration of a tryptaminergic psychedelic drug, such as, but not limited to, psilocybin every second day for 4 weeks to patients with chronic schizophrenia or another psychiatric disorder such as treatment resistant depression or PTSD that does not respond to conventional therapy at a dose that is insufficient to induce psychedelic effects concurrently with clozapine, initiated at least two weeks prior to the inception of treatment with psilocybin.

FIG. 9 schematically depicts another envisioned treatment regimen comprising administration of D-serine at a dose of at least 30 mg/kg and psilocybin at a dose of 0.02-0.04 mg/kg, where D-serine is administered daily (continuous line) for two weeks prior to inception of psilocybin which is then administered every second day (broken line) for 4 weeks concurrently with D-serine, for the treatment of subjects with chronic schizophrenia, treatment-resistant depression or PTSD that does not respond to conventional therapy (referred to as regular treatment in FIG. 9).

### DETAILED DESCRIPTION OF THE INVENTION

This invention provides, in some embodiments, novel treatment approaches for treating schizophrenia. This invention provides, in some embodiments, novel treatment approaches for chronic schizophrenia patients with negative symptoms, or novel treatment approaches for patients with post-traumatic stress disorder or novel treatment approaches for depression, and in some aspects, the invention specifically contemplates treating treatment-resistant cases.

In some aspects of the invention, the invention provides for concurrent treatment agents modulating 5-HT2A receptor function, which can induce psychological effects in the short term with neuroplastic changes in key brain areas in the longer terms, specifically and exceptionally through the combinations described herein.

In other aspects, the invention contemplates combinations/compositions/kits for use that contain antipsychotic drugs that can improve negative symptoms and cognitive function in patients with chronic schizophrenia, for example, such as clozapine. According to this aspect, and uniquely herein, it is now demonstrated for the first time that combinations of a psychedelic, such as psilocybin (PSIL) and an antipsychotic that possesses 5-HT2A antagonist properties, such as clozapine (CLZ), prevents the induction of positive symptoms of schizophrenia, in animal models approximating the disease, supporting the potential for the described combinations to reduce negative symptoms of schizophrenia, as well.

In other aspects, the invention contemplates combinations/compositions/kits for use that contain NMDAR agonists or at least partial agonists. According to this aspect, it is now for the first time contemplated making use of an NMDAR agonist, such as D-serine, to elicit clinical improvements in negative symptoms and overall symptomatology in patients with chronic schizophrenia, as part of the combination therapies described herein.

In some embodiments, the combinations of this invention prevent the psychedelic effects of the psychedelic drug, while at the same time allowing the neuroplastic effect of the psychedelic drug. According to this aspect and in some embodiments, such combinations therefore improve negative symptoms of schizophrenia.

In some aspects, the invention specifically contemplates use of agents that modulate the function of NMDA receptors in the cortex and other brain areas in combination with psychedelics and/or agents modulating 5-HT2A receptor function and in some embodiments, it is specifically contemplated to use agents modulating 5-HT2A receptor function and psychedelics for their effect on treating schizophrenia and other neuropsychiatric diseases.

In one embodiment, this invention provides a combination therapy comprising a psychedelic drug and an NMDA receptor agonist.

In some embodiments, this invention provides a combination therapy comprising a psychedelic drug and an antipsychotic possessing 5-HT2A antagonist properties and in some aspects, optionally in addition to an NMDA receptor agonist or partial agonist.

In other aspects, the combination regimens/treatments of this invention making use of a psychedelic drug (such as but not limited to psilocybin) and NMDAR agonists/partial agonists, (such as, but not limited to D-serine) for the treatment of schizophrenia and other neuropsychiatric diseases, provides another unique and highly advantageous combination therapy.

In some embodiments, this invention provides a combination therapy comprising an NMDA receptor agonist or partial agonist and a psychedelic drug, which in some embodiments, is a serotonergic psychedelic, such as, but not limited to a serotonin 5-HT2A receptor agonist.

According to this aspect and in some embodiments, the use of a serotonergic psychedelic (serotonin 5-HT2A receptor agonists) is contemplated for the combinations, compositions, and kits of this invention, or for use in the methods or therapeutic uses as herein described, which in some aspects, may include:

Tryptamines (such as, in some embodiments, alkylated tryptamines]) such as Psilocin, also known as ‘4-HO-DMT’; Psilocybin, also known as ‘4-PO-DMT’; the primary active constituent of the Psilocybe genus of mushrooms; its effects are mostly attributed to psilocin, to which it is a prodrug via dephosphorylation; Bufotenin, also known as ‘5-HO-DMT’ and dimethylserotonin; another constituent of the skin and venom of psychoactive toads, also a metabolite of 5-MeO-DMT; Bacocystin, also known as ‘4-PO-NMT’; another active constituent of the Psilocybe genus of mushrooms; Aeruginascin, also known as ‘4-PO-N-TMT’, an active constituent of the mushroom Inocybe aeruginascens; 5-MeO-DMT, the primary active constituent of the skin and venom of psychoactive toads, a prodrug to bufotenin via demethylation; N,N-Dimethyltryptamine, also known as ‘DMT’; the primary active constituent of the Amerindian brew ayahuasca; endogenously present in various plants and animals, including humans; 5-Bromo-DMT, found in the marine invertebrates Smenospongia aurea and Smenospongia echina, as well as in Verongula rigida; N-Methyl-N-ethyltryptamine, also known as ‘MET’; N-Methyl-N-isopropyltryptamine, also known as ‘MiPT’; N-Methyl-N-propyltryptamine, also known as ‘MPT’; N,N-Diethyltryptamine, also known as ‘DET’; N-Ethyl-N-isopropyltryptamine, also known as ‘EiPT’; N-Methyl-N-butyltryptamine, also known as ‘MBT’; N-Propyl-N-isopropyltryptamine, also known as ‘PiPT’; N,N-Dipropyltryptamine, also known as ‘DPT’,
N,N-Diisopropyltryptamine, also known as ‘DiPT’; N,N-Diallyltryptamine, also known as ‘DALT’; N,N-Dibutyltryptamine, also known as ‘DBT’; N-Ethyltryptamine, also known as ‘NET’; N-Methyltryptamine, also known as ‘NMT’; Trimethyltryptamine, also known as ‘TMT’ (2,N,N-TMT, 5,N,N-TMT, and 7,N,N-TMT); α-Methyltryptamine, also known as ‘YMT’ and ‘AMT’; also has entactogenic properties; α-Ethyltryptamine, also known as ‘αET’ and ‘AET’; also has entactogenic properties; α,N-DMT; α,N,N-Trimethyltryptamine, also known as ‘α-TMT; Ethocybin, also known as ‘4-PO-DET’, ‘CEY-19’, and ‘CEY-39’; 4-HO-MET, also known as ‘Metocin’, ‘Methylcybin’, and ‘Colour’; 4-HO-DET, also known as ‘Ethocin’ and ‘CZ-74’; 4-HO-MPT, also known as ‘Meprocin’; 4-HO-MiPT, also known as ‘Miprocin’; 4-HO-MALT; 4-HO-DPT, also known as ‘Deprocin’; 4-HO-DiPT, also known as ‘Iprocin’; 4-HO-DALT, also known as ‘Daltocin’; 4-HO-DBT; 4-HO-DSBT; 4-HO-αMT; 4-HO-MPMI, also known as ‘Lucigenol’; 4-HO-TMT; 4-HO-1,N,N-TMT, also known as ‘1-Me-4-HO-DMT’ and ‘1-methylpsilocin’; 4-HO-5-MeO-DMT, also known as ‘Psilomethoxin’; 4-AcO-DMT, also known as ‘psiloacetin’; its effects are partially attributed to psilocin, to which it is a prodrug via deacetylation; 4-AcO-MET, also known as ‘Metacetin’; 4-AcO-MiPT; 4-AcO-MALT; 4-AcO-DET, also known as ‘Ethacetin’; 4-AcO-EiPT, also known as ‘Ethipracetin’; 4-AcO-DPT, also known as ‘Depracetin’; 4-AcO-DiPT, also known as ‘Ipracetin’; 4-AcO-DALT, also known as ‘Daltacetin’; 4-MeO-DMT; 4-MeO-MiPT; 5-MeO-NMT; 5-MeO-MET; 5-MeO-MPT; 5-MeO-MiPT, also known as ‘Moxy’; also has entactogenic properties; 5-MeO-MALT; 5-MeO-DET; 5-MeO-EiPT; 5-MeO-EPT; 5-MeO-PiPT; 5-MeO-DPT; 5-MeO-DiPT, also known as ‘Foxy Methoxy’; 5-MeO-DALT; 5-MeO-αMT, also has entactogenic properties; 5-MeO-αET, also has entactogenic properties; 5-MeO-MPMI; 5-MeO-2,N,N-TMT, also known as ‘Indomethacin’ and ‘Indapex’; 5-MeO-7,N,N-TMT; 5-MeO-α,N-DMT, also known as ‘α,N,O-TMS’; 4-F-5-MeO-DMT; 5-MeS-DMT; 5-Me-MiPT; 5-HO-DiPT; 2-α-DMT; 2-Me-DET; 4-Me-αMT; 4-Me-αET, also has entactogenic properties; 7-Me-αET, also has entactogenic properties; 4,5-DHP-AMT, also known as ‘AL-37350A’; 4,5-DHP-DMT; 4,5-MDO-DMT; 4,5-MDO-DiPT; 5,6-MDO-DiPT; 5,6-MDO-MiPT; 5-Fluoro-αMT, also has entactogenic properties; 6-Fluoro-αMT; 6-Fluoro-DMT; N,N-Tetramethylenetryptamine, also known as ‘Pyr-T’; 4-HO-pyr-T; 5-MeO-pyr-T; RU-28306, also known as ‘4,a-Methylene-N,N-DMT’; O-4310, also known as ‘6-Fluoro-1-Isopropyl-4-HO-DMT’; CP-132,484, also known as ‘4,5-DHP-1-Methyltryptamine’, or combinations thereof.

In some embodiments, the serotonergic psychedelics (serotonin 5-HT2A receptor agonists) contemplated for the combinations, compositions, and kits of this invention, or for use in the methods or therapeutic uses as herein described, may include:

Benzofuran derivatives; Dimemebfe, also known as ‘5-MeO-BFE’; 5-MeO-DiBF; Ibogoids; Ibogaine, the primary active constituent of iboga rootbark; also has dissociative properties; Voacangine, another active constituent of iboga rootbark; Ergolines; Lysergic acid diethylamide, also known as ‘LSD’ and ‘acid’; Lysergic acid amide, also known as ‘LSA’ and ‘ergine’; the primary active constituent of morning glory and Hawaiian baby woodrose seeds; N1-Methyl-lysergic acid diethylamide, also known as ‘MLD-41’; N-Acetyl-lysergic acid diethylamide, also known as ‘ALD-52’; 1-Propionyl-lysergic acid diethylamide, also known as ‘1P-LSD’; its effects are partially attributed to LSD, to which it is a prodrug via hydrolyzation; 1-cyclopropanoyl-d-lysergic acid diethylamide, also known as ‘1cP-LSD’; 1-valeryl-D-lysergic acid diethylamide, also known as ‘IV-LSD’; 6-Allyl-6-nor-lysergic acid diethylamide, also known as ‘AL-LAD’; 6-Butyl-6-nor-lysergic acid diethylamide, also known as ‘BU-LAD’; 6-Ethyl-6-nor-lysergic acid diethylamide, also known as ‘ETH-LAD’; 1-Propionyl-6-Ethyl-6-nor-lysergic acid diethylamide, also known as ‘1P-ETH-LAD’; 6-Propyl-6-nor-lysergic acid diethylamide, also known as ‘PRO-LAD’; 6-Cyclopropyl-6-nor-lysergic acid diethylamide, also known as ‘CYP-LAD’; 6-nor-Lysergic acid diethylamide, also known as ‘PARGY-LAD’; Lysergic acid ethylamide, also known as ‘LAE-32’; Lysergic acid?-hydroxyethylamide, also known as ‘LSH’ and ‘LAH’; another active constituent of morning glory seeds; an active constituent of some species of fungi; Lysergic acid 2-butyl amide, also known as ‘LSB’; Lysergic acid 3-pentyl amide, also known as ‘LSP’; Lysergic acid methyl ester, also known as ‘LSME’; Lysergic acid 2,4-dimethylazetidide, also known as ‘LSZ’ and ‘LA-SS-Az’; Lysergic acid piperidine, also known as ‘LSD-Pip’; N,N-Dimethyl-lysergamide, also known as ‘DAM-57’; Methylisopropyllysergamide, also known as ‘MIPLA’; N,N-Diallyllysergamide, also known as ‘DAL’; N-Pyrrolidyllysergamide, also known as ‘LPD-824’; N-Morpholinyllysergamide, also known as ‘LSM-775’; 1-methyl-lysergic acid butanolamide, also known as ‘Methysergide’; the active constituent of Sansert and Deseril; a prodrug which has to be metabolized to methylergometrine to become psychoactive; Lysergic acid?-propanolamide, also known as ‘Ergonovine’ and ‘Ergometrine’; another active constituent of morning glory seeds, and an active constituent of ergot fungi’ Lysergic acid 1-butanolamide, also known as ‘Methylergonovine’, ‘Methergine’, and ‘Methylergometrine’; another active constituent of morning glory seeds and of ergot fungi; Phenethylamines (more specifically alkoxylated phenethylamines); or combinations thereof.

In some embodiments, the serotonergic psychedelics (serotonin 5-HT2A receptor agonists) contemplated for the combinations, compositions, and kits of this invention, or for use in the methods or therapeutic uses as herein described, may include:

Substituted phenethylamines; Mescaline, the primary active constituent of certain cacti, such as peyote and San Pedro; Lophophine, also known as ‘MMDPEA’; another active constituent of certain cacti, such as peyote and San Pedro; also has entactogenic properties; Isomescaline; Cyclopropylmescaline; Thioisomescaline (2-TIM, 3-TIM, and 4-TIM); 4-Desoxymescaline; Jimscaline; Escaline; Metaescaline; Thiometaescaline (3-TME, 4-TME, and 5-TME); Trisescaline; Thiotrisescaline (3-T-TRIS and 4-T-TRIS); Symbescaline; Asymbescaline; Thiosymbescaline (3-TSB and 4-TSB); Phenescaline; Allylescaline, also known as ‘AL’; Methallylescaline; Proscaline; Isoproscaline; Metaproscaline; Thioproscaline; Buscaline; Thiobuscaline; «-ethylmescaline, also known as ‘AEM’; Ariadne, also known as ‘α-Et-DOM’, ‘4C-D’, and ‘Dimoxamine’; Macromerine; MEPEA; TOM (2-TOM and 5-TOM); Bis-TOM; TOMSO, also known as ‘2-methoxy-4-methyl-5-methylsulfinylamphetamine’; TOET (2-TOET and 5-TOET); BOH; BOM, also known as ‘β-Methoxy-mescaline’; β-D; 4-D; DME; F-2; F-22; FLEA, also known as ‘MDHMA’; MDPH; MDMP; Propynyl; 2C family (2,5-dimethoxy, 4-substituted phenethylamines); βk-2C-B; 2C-B; 2CB-2EtO; 2CB-5EtO; 2CB-diEtO; 2C-B-FLY; 2C-B-BUTTERFLY; 2C-C; 2C-D; 2CD-2EtO; 2CD-diEtO; 2CD-5EtO; 2C-E; 2C-EF; 2C-F; 2C-G (2C-G-1, 2C-G-2, 2C-G-3, 2C-G-4, 2C-G-5, 2C-G-6, and 2C-G-N); 2C-H; 2C-I; 2CI-2EtO; 2C-iP; 2C-N; 2C-O; 2C-O-4; 2C-P; 2C-SE; 2C-T; 2CT-5EtO; 2C-T-2; 2CT-2-2EtO; 2CT-2-5EtO; 2CT-2-diEtO; 2C-T-4 (2C-T-4 and ?-2C-T-4); 2CT-4-2EtO; 2C-T-7; 2CT-7-2EtO; 2C-T-8; 2C-T-9; 2C-T-13; 2C-T-15; 2C-T-16; 2C-T-17; 2C-T-19; 2C-T-21; 2C-TFM; 2C-YN; BOB, also known as ‘β-Methoxy-2C-B’; BOD, also known as ‘β-Methoxy-2C-D’; BOHD, also known as ‘β-Hydroxy-2C-D’; HOT-2; HOT-7; HOT-17; Indane derivatives; 2CB-Ind; Benzocyclobutene derivatives; 2C-BCB, also known as ‘TCB-2’; NBOMe derivatives; NBOMe-mescaline; 2C-H-NBOMe, also known as ‘25H-NBOMe’; 2C-C-NBOMe, also known as ‘25C-NBOMe’; 2CBCB-NBOMe, also known as ‘NBOMe-TCB-2’; 2CBFly-NBOMe, also known as ‘Cimbi-31’; 2C-B-NBOMe, also known as ‘25B-NBOMe’, ‘M25B-NBOMe’, ‘BOM 2-CB’, ‘Cimbi-36’, ‘Nova’, or ‘New Nexus’; 2C-I-NBOM, also known as ‘25I-NBOMe’, ‘Cimbi-5’, “Solaris”, or “N-Bomb”; 2C-TFM-NBOMe, also known as ‘25TFM-NBOMe’; 2C-D-NBOMe, also known as ‘25D-NBOMe’; 2C-G-NBOMe, also known as ‘25G-NBOMe’; 2C-E-NBOMe, also known as ‘25E-NBOMe’; 2C-P-NBOMe, also known as ‘25P-NBOMe’; 2C-iP-NBOMe, also known as ‘25iP-NBOMe’; 2C-CN-NBOMe, also known as ‘25CN-NBOMe’; 2C-N-NBOMe, also known as ‘25N-NBOMe’; 2C-T-NBOMe, also known as ‘25T2-NBOMe’; 2C-T-4-NBOMe, also known as ‘25T4-NBOMe’; 2C-T-7-NBOMe, also known as ‘25T7-NBOMe’; DMBMPP. 2-Benzylpiperidine analogue of 25B-NBOMe; NBOH derivatives; 2C-C-NBOH, also known as ‘25C-NBOH’ and ‘NBOH-2CC’; 2C-B-NBOH, also known as ‘25B-NBOH’; 2C-I-NBOH, also known as ‘25I-NBOH’; 2C-CN-NBOH, also known as ‘25CN-NBOH’ and ‘NBOH-2C-CN’; NBMD derivatives; 2C-I-NBMD, also known as ‘25I-NBMD’; NBF derivatives; 2C-C-NBF, also known as ‘25C-NBF’; 2C-B-NBF, also known as ‘25B-NBF’; 2C-I-NBF, also known as ‘25I-NBF’; Substituted amphetamines (alpha-methyl-phenethylamines); 3C family (3,5-dimethoxy, 4-substituted amphetamines); 3C-E; 3C-P; 3C-DFE; 3C-BZ; DOx family (2,5-dimethoxy, 4-substituted amphetamines); DOAM; DOB; Meta-DOB; Methyl-DOB; DOBU; DOC; DOEF; DOET, also known as ‘DOE’; DOI′ DOM, also known as ‘STP’; Ψ-DOM; DON; DOPR; DOiPR; DOT, also known as ‘Aleph’ (Aleph-2, Aleph-4, Aleph-6, and Aleph-7); Meta-DOT; Ortho-DOT; DOTFM; Phenylcyclopropylamine derivatives; DMCPA; DMMDA; DMMDA-2; 2,5-dimethoxy-3,4-dimethylamphetamine, also known as ‘Ganesha’; (G-3, G-4, G-5, and G-N); 4-methyl-2,5-dimethoxymethamphetamine, also known as ‘Beatrice’, and ‘MDO-D’, ‘MDOM’; 2,N-dimethyl-4,5-methylenedioxyamphetamine, also known as ‘Madam-6’; Dimethoxyamphetamine (2,4-DMA, 2,5-DMA, and 3,4-DMA); Trimethoxyamphetamine (TMA-2, TMA-6); Tetramethoxyamphetamine; Br-DragonFLY; TFMFly; 2-Bromo-4,5-methylenedioxyamphetamine; 4-Bromo-3,5-dimethoxyamphetamine; EEE; EEM; EME; EMM; EDMA; EIDA; Ethyl-J, also known as ‘EBDB’; Methyl-J, also known as ‘MDMB’; Ethyl-K, also known as ‘EBDP’; Methyl-K, also known as ‘MBDP’ and ‘UWA-91’; IDNNA; Iris; MDAI; MDMAI; MDAT; MDMAT; MDAL; MDBU; MDBZ; MDDM; MDIP; MDMEOET; MDMEO; MDOH, also known as ‘MDH’; MDHOET; MDPL; MDCPM; MDPR; MEDA; MEM; Methyl-DMA; MDA, also known as ‘3-methoxy-MDA’ (2T-MMDA-3a and 4T-MMDA-2); MMDA-2; 5-Methyl-MDA; MEE; MME; MPM; DiFMDA; 5-APB; 6-APB, also known as ‘Benzofury’; 5-APDB; 6-APDB; 5-MAPB; 5-MAPDB; 6-MAPDB; 6-MAPB; 6-EAPB; 5-EAPB; Para-Methoxyamphetamine, also known as ‘PMA’ and ‘4-MA’; Paramethoxymethamphetamine, also known as ‘PMMA’, ‘Methyl-MA’, and ‘4-MMA’; 4-Ethylamphetamine, also known as ‘4-EA’; 3-Methoxy-4-methylamphetamine, also known as ‘MMA’; 4-Methylmethamphetamine, also known as ‘4-MMA’; 4-Methylthioamphetamine, also known as ‘4-MTA’; 4-Fluoroamphetamine, also known as ‘4-FA’, ‘PAL-303’, ‘Flux’, ‘Flits’, ‘R2D2’, and ‘Miley’; Norfenfluramine, also known as ‘3-TFMA’; Para-Iodoamphetamine, also known as ‘PIA’, ‘4-iodoamphetamine’, and ‘4-IA’; Para-Chloroamphetamine, also known as ‘PCA’, ‘4-chloroamphetamine’, and ‘4-CA’; Benzoxazines (for example, cyclopropylethynylated benzoxazines); Substituted benzoxazines; Efavirenz, the active constituent of Sustiva, Stocrin, and Efavir; or any combinations thereof.

In some aspects, the combinations/compositions/kits/methods/uses contemplated specifically describe inclusion of psychedelics, specifically contemplating psilocybin in this context.

As used herein, the term “psilocybin” refers to any pharmaceutically active form of same including metabolites of same such as psilocin, and the two are to be considered as interchangeable such that specific mention of “psilocybin” will equally refer to “psilocin” and vice versa and may be appreciated herein as PSIL, as well.

In some embodiments, the combinations/compositions/kits/methods/uses are contemplated wherein same are provided to a subject in need thereof, wherein a therapeutically effective amount of an NMDAR agonist or partial agonist, is provided further in combination with a psychedelic drug, such as that described in US2009203750-A1, AU-2005300045-A1, AU-2009214724-A1, US2019201402-A1, WO-2022067165-A1, U.S. Pat. No. 11,254,640-B2, EP-3519816-A1, WO-2021076572-A1 or WO-2020176599-A1, which are hereby incorporated by reference in their entirety

In some embodiments, the combinations/compositions/kits/methods and uses of this invention include a psychedelic drug and an antipsychotic possessing 5-HT2A antagonist properties. According to this aspect and in some embodiments, the antipsychotic possessing 5-HT2A antagonist properties is clozapine, olanzapine, quetiapine, risperidone, aripiprazole, asenapine, pizotifen, ziprasidone, chloropromazine, thioridazine, paloperidone, iloperidone, lurasidone, chlorprothixene, sertindole, lumateperone, zuclopenthixol, loxapine, amisulpiride, thiothixene, zotepine, cariprazine or a combination thereof and the combined use of same with a psychedelic drug including any embodiment, listed herein regarding any serotonergic psychedelic as herein described is to be considered as part of this invention.

In some embodiments, the antipsychotic possessing 5-HT2A antagonist properties is clozapine.

In some embodiments, the clozapine is provided at a dosage of 100-400 mg/day, or in some embodiments, the clozapine is provided at a dosage of 100-200 mg/day, or in some embodiments, the clozapine is provided at a dosage of 150-250 mg/day, or in some embodiments, the clozapine is provided at a dosage of 175-275 mg/day, or in some embodiments, the clozapine is provided at a dosage of 200-300 mg/day, or in some embodiments, the clozapine is provided at a dosage of 250-350 mg/day, or in some embodiments, the clozapine is provided at a dosage of 275-375 mg/day, or in some embodiments, the clozapine is provided at a dosage of 300-400 mg/day.

In some aspects, the specific inclusion of clozapine at a dosage of from about 100-400 mg/day, or any dosage range therein as described herein, results in the blockade of negative effects of the psychedelic effects, while concurrently providing for the influence of the positive neuroplasticity effects of the psychedelic alone.

In some embodiments, the combinations/compositions/kits/methods and uses of this invention include a psychedelic drug and an NMDAR agonist or partial agonist.

In some embodiments, the combinations/compositions/kits/methods and uses of this invention include an NMDAR agonist or a partial agonist and an antipsychotic possessing 5-HT2A antagonist properties.

In some embodiments, the NMDA receptor agonist is D-serine.

In some embodiments, the D-serine is administered at any appropriate dosage, for example, at a dosage of from 1.5-15.0 grams/day, which in some embodiments, may be at a dosage of from 5-15 grams/day. In other embodiments, the D-serine is administered at a dosage of from about 1.5-7 grams/day, or in some embodiments, the D-serine is administered at a dosage of from about 3-10 grams/day, or in some embodiments, the D-serine is administered at a dosage of from about 5-15 grams/day, or in some embodiments, the D-serine is administered at a dosage of from about 7-15 grams/day, or in some embodiments, the D-serine is administered at a dosage of from about 10-15 grams/day.

It will be appreciated, that depending on the route of administration, formulation used, or application being addressed, a different dosage may be what is therapeutic and the invention will therefore not be bound by or otherwise limited to this dosage alone, but rather, same represents a guidance to address some embodied therapeutic amounts for use in accordance with the invention.

In some embodiments, the NMDA receptor agonist is glycine or in some embodiments, the NMDA receptor agonist is Alanine (D-alanine, L-alanine), or in some embodiments, the NMDA receptor agonist is Milacemide, or in some embodiments, Sarcosine (monomethylglycine), or in some embodiments, the NMDA receptor agonist is L-Serine.

In some embodiments, the NMDA receptor agonist is 3,5-Dibromo-L-phenylalanine, Aminocyclopropanecarboxylic acid (ACC), or rapastinel (GLYX-13) or apimostinel (NRX-1074), or HA-966, or Homoquinolinic, or Zelquistinel (AGN-241751), or Apimostinel (NRX-1074) or others known in the art.

In some embodiments, the NMDA receptor agonist is D-cycloserine.

In some embodiments, the D-cycloserine is administered at any appropriate dosage, for example, at a dosage of from 100-1500 milligrams/day. In some embodiments, the D-cycloserine is administered at a dosage of from 250-1500 mg/day, or in some embodiments, the D-cycloserine is administered at a dosage of from 200-500 mg/day, or in some embodiments, the D-cycloserine is administered at a dosage of from 300-700 mg/day, or in some embodiments, the D-cycloserine is administered at a dosage of from 350-1500 mg/day, or in some embodiments, the D-cycloserine is administered at a dosage of from 400-800 mg/day, or in some embodiments, the D-cycloserine is administered at a dosage of from 450-1500 mg/day, or in some embodiments, the D-cycloserine is administered at a dosage of from 500-900 mg/day, or in some embodiments, the D-cycloserine is administered at a dosage of from 550-1000 mg/day, or in some embodiments, the D-cycloserine is administered at a dosage of from 700-1500 mg/day, or in some embodiments, the D-cycloserine is administered at a dosage of from 1000-1500 mg/day, or any sub-range described herein.

It will be appreciated, that depending on the route of administration, formulation used, or application being addressed, a different dosage may be what is therapeutic and the invention will therefore not be bound by or otherwise limited to this dosage alone, but rather, same represents a guidance to address some embodied therapeutic amounts for use in accordance with the invention.

In some embodiments, deuterated forms of the amino acids described herein are specifically contemplated for use. For example, and in some embodiments, the deuterated form of D-serine is specifically envisioned for incorporation in the combinations/compositions/kits/methods and uses of this invention.

In other embodiments, the glycine reuptake inhibitor bitopertin is specifically envisioned for use. For example, and in some embodiments, the glycine reuptake inhibitor bitopertin is specifically envisioned for incorporation in the combinations/compositions/kits/methods and uses of this invention.

In other embodiments, the invention specifically relates to a composition comprising an NMDAR agonist and a psychedelic drug, or a treatment regimen combining the administration of an NMDAR agonist and a psychedelic drug to a subject, or a combination therapy comprising the administration of an NMDAR agonist and a psychedelic drug.

According to this aspect, and in some embodiments, the NMDAR agonist is D-Serine, including in any embodiment of same as herein described.

Thus, in some embodiments, the invention specifically relates to a composition comprising D-Serine and a psychedelic drug, or a treatment regimen combining the administration of D-Serine and a psychedelic drug to a subject, or a combination therapy comprising the administration of D-Serine and a psychedelic drug. According to this aspect and in some embodiments, the psychedelic drug is psilocybin.

Thus, in some embodiments, the invention specifically relates to a composition comprising D-cycloserine and a psychedelic drug, or a treatment regimen combining the administration of D-cycloserine and a psychedelic drug to a subject, or a combination therapy comprising the administration of D-cycloserine and a psychedelic drug. According to this aspect and in some embodiments, the psychedelic drug is psilocybin.

In still other embodiments, the invention specifically relates to a composition comprising an antipsychotic possessing 5-HT2A antagonist properties and a psychedelic drug, or a treatment regimen combining the administration of an antipsychotic possessing 5-HT2A antagonist properties and a psychedelic drug to a subject, or a combination therapy comprising the administration of an antipsychotic possessing 5-HT2A antagonist properties and a psychedelic drug. According to this aspect and in some embodiments, the psychedelic drug is psilocybin and the antipsychotic possessing 5-HT2A antagonist properties is clozapine.

According to these aspects, it will be understood that each of the above-described compositions will be formulated for daily administration, or in some embodiments, for bolus administration, or other convenient administration means.

In some embodiments, the clozapine is administered at any appropriate dosage, for example, at a dosage of from 100-400 milligrams/day, or any of the embodied ranges described herein. It will be appreciated, that depending on the route of administration, formulation used, or application being addressed, a different dosage may be what is therapeutic and the invention will therefore not be bound by or otherwise limited to this dosage alone, but rather, same represents a guidance to address some embodied therapeutic amounts for use in accordance with the invention.

In some embodiments, the psilocybin is according to recommended micro dosages for daily administration, and in some embodiments, typical adult dosages are envisioned for use with non-daily treatment regimens. It will be appreciated, that depending on the route of administration, formulation used, or application being addressed, a different dosage may be therapeutic and the invention will therefore not be bound by or otherwise limited to this dosage alone, but rather, same represents a guidance to address some embodied therapeutic amounts for use in accordance with the invention.

In some embodiments, the combinations/compositions/kits/methods and uses of this invention include a psychedelic drug and an alanine-serine cysteine transporter inhibitor, a D-amino acid oxidase inhibitor, a glycine transport inhibitor or a combination thereof.

In other embodiments, the combinations/compositions/kits/methods and uses of this invention include an alanine-serine-cysteine transporter inhibitor, a D-amino acid oxidase inhibitor, a glycine transport inhibitor and an antipsychotic possessing 5-HT2A antagonist properties.

In some aspects of the invention, the combinations/compositions/kits/methods/uses contemplate the combined use of the active agents described herein for treating, reducing the incidence of, reducing the severity of or improving a pathology of a subject with a neuropsychiatric disease or disorder, neurologic disease or disorder, a neurocognitive disease or disorder, or a motor disease or disorder.

In some aspects, the disease or disorder is schizophrenia, depression, post-traumatic stress disorder, obsessive-compulsive disorder (OCD), cerebral palsy, muscular dystrophy, spina bifida, spinal muscle atrophy (SMA), Parkinson's disease, epilepsy, amyotrophic lateral sclerosis (ALS), Ataxia, attention-deficit hyperactivity disorder, generalized anxiety disorder, or panic disorder, cervical dystonia, general dystonia, chorea, functional movement disorder, Huntington's disease, multiple system atrophy, myoclonus, chronic pain, inflammation, Parkinsonism, Alzheimer's disease, sleep-wake disorders, Stroke or repeated head trauma, progressive supranuclear palsy, restless legs syndrome, tardive dyskinesia, Tourette syndrome, tremor, or Wilson's disease or an autism spectrum disorder (ASD) or a symptom thereof, or any related disorder as herein described.

In some aspects of the invention, the combinations/compositions/kits/methods/uses contemplate the combined use of the active agents described herein for the treatment of Schizophrenia, and in some embodiments, in particular, treating the negative symptoms of Schizophrenia, or in some aspects, treating treatment-resistant Schizophrenia.

In some aspects of the invention, the combinations/compositions/kits/methods/uses contemplate the combined use of the active agents described herein for treating, reducing the incidence of, reducing the severity of or improving a pathology of a subject with schizophrenia comprises reducing or abrogating negative symptoms of schizophrenia.

According to these aspects and in some embodiments, the combinations/compositions/kits/methods/uses contemplate the combined use of a psychedelic drug, including in any embodiment as herein described and an antipsychotic possessing 5-HT2A antagonist properties, as well, specifically contemplating any embodiment of same as herein described.

In some embodiments, according to these aspects, the psychedelic is psilocybin, Dimethyltryptamine (DMT), 5-Methoxy-N,N-dimethyltryptamine (5-MeO-DMT), Mescaline, 3,4-methylenedioxymethamphetamine (MDMA) or Lysergic acid diethylamide (LSD) or a combination thereof and the antipsychotic possessing 5-HT2A antagonist properties is clozapine, olanzapine, quetiapine, risperidone, aripiprazole, asenapine, pizotifen, ziprasidone, chloropromazine, thioridazine, paloperidone, iloperidone, lurasidone, chlorprothixene, sertindole, lumateperone, zuclopenthixol, loxapine, amisulpiride, thiothixene, zotepine, cariprazine or a combination thereof.

In further embodiments, according to these aspects, the psychedelic is psilocybin, and the antipsychotic possessing 5-HT2A antagonist properties is clozapine.

In some embodiments, according to these aspects, the treatment may be stand-alone or as an add-on to other drugs or adjuvants given in treatment sessions during various forms of psychotherapy. Thus, the treatments can be given daily or by boluses at one-to-three-week intervals.

In some aspects of the invention, the combinations/compositions/kits/methods/uses contemplate the combined use of the active agents described herein for treating, reducing the incidence of, reducing the severity of or improving a pathology of a subject with post-traumatic stress disorder.

In some aspects of the invention, the combinations/compositions/kits/methods/uses contemplate the combined use of the active agents described herein for treating, reducing the incidence of, reducing the severity of or improving a pathology of a subject with depression, which in some embodiments is treatment-resistant depression.

In some aspects of the invention, the combinations/compositions/kits/methods/uses contemplate the combined use of the active agents described herein for the treatment of enhancing neuroplasticity in a subject with schizophrenia, depression or post-traumatic stress disorder.

In some aspects, the combinations/compositions/kits/methods/uses contemplate the combined use of the active agents described herein for the treatments as described hereinabove, make use of a psychedelic drug and an antipsychotic possessing 5-HT2A antagonist properties, further comprising an NMDAR agonist. In some aspects, the psychedelic drug may typically produce certain adverse events, which are reduced, or abrogated or mitigated, as part of the combination of agents used in conjunction with same.

In some aspects, as used herein, the following Medical Dictionary for Regulatory Activities (MedDRA) terms are considered to be adverse events that are psychedelic in nature: altered mood, altered state of consciousness, autoscopy, delusional perception, disinhibition, dissociation, dissociative identity disorder, dreamy state, emotional disorder, euphoric mood, feeling abnormal, hallucination, hyperacusis, hyperaesthesia, hypoaesthesia, illusion, paranoia, parosmia, photophobia, sensory disturbance, time perception altered, thinking abnormal, synaesthesia, substance-induced psychotic distress, and somatic hallucination. In some aspects, the combinations/compositions/kits/methods/uses contemplate the combined use of the active agents described herein for the treatments as described hereinabove, will exhibit at least a reduction or lessening or other improvement in these adverse events described herein.

As used herein, a therapy or therapeutic that is administered “concurrently” with another drug is administered, in some aspects, within 1 day of the other drug. In some embodiments, a therapy or therapeutic that is administered concurrently with another drug is administered at about the same time, within about 5 minutes, within about 10 minutes, within about 15 minutes, within about 20 minutes, within about 30 minutes, within about 45 minutes, within about 1 hour, within about 2 hours, within about 3 hours, within about 4 hours, within about 5 hours, within about 6 hours, within about 7 hours, within about 8 hours, within about 9 hours, within about 10 hours, within about 11 hours, within about 12 hours, within about 13 hours, within about 14 hours, within about 15 hours, within about 16 hours, within about 17 hours, within about 18 hours, within about 19 hours, within about 20 hours, within about 21 hours, within about 22 hours, within about 23 hours, or within about 24 hours of administration of the other drug.

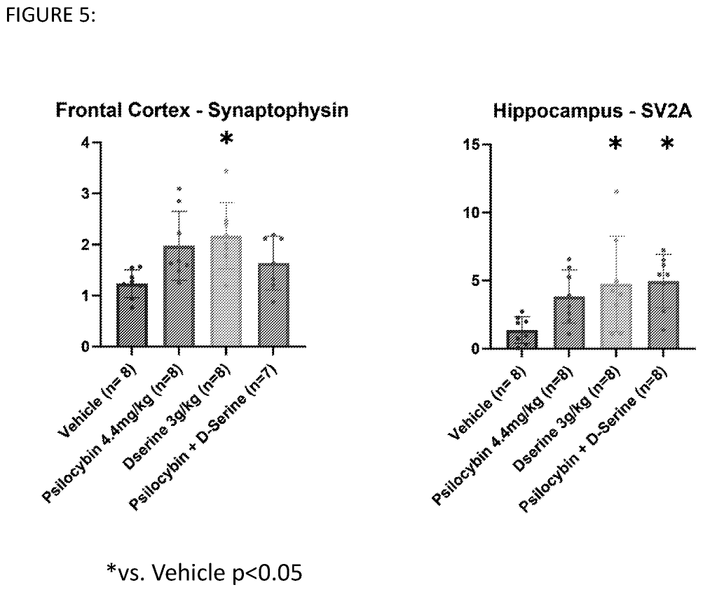

In some embodiments, the combinations/compositions/kits/methods/uses contemplate the combined use of an NMDAR agonist, or partial agonist and a psychedelic drug or an antipsychotic possessing 5-HT2A antagonist properties, or a combination thereof may follow an administration schedule such as that described in FIG. 5, herein, or any similar appropriate staggered regimen as herein described.

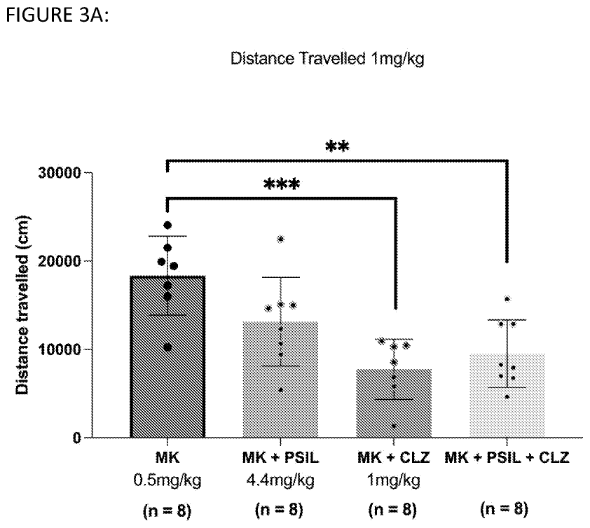

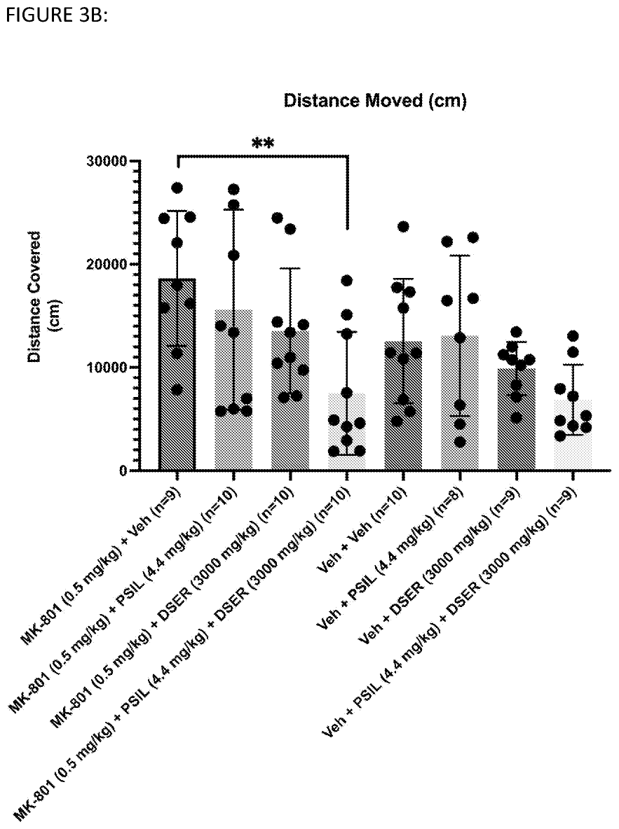

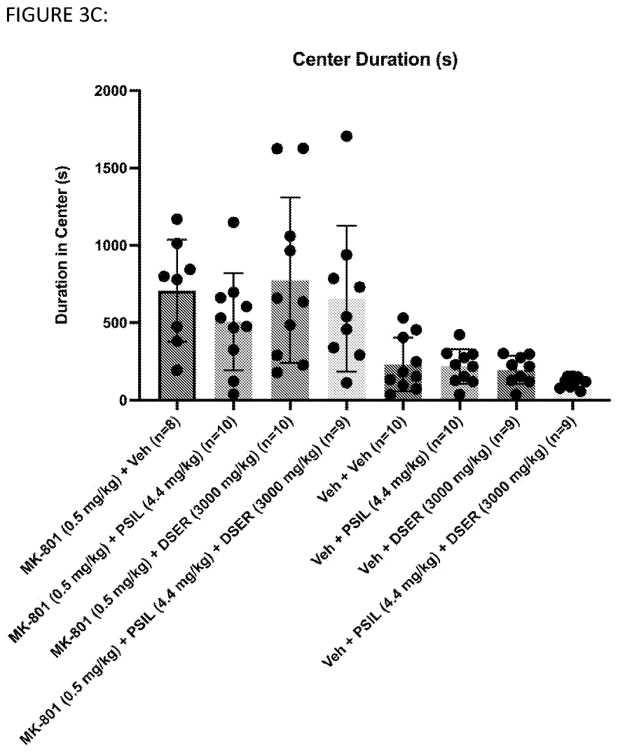

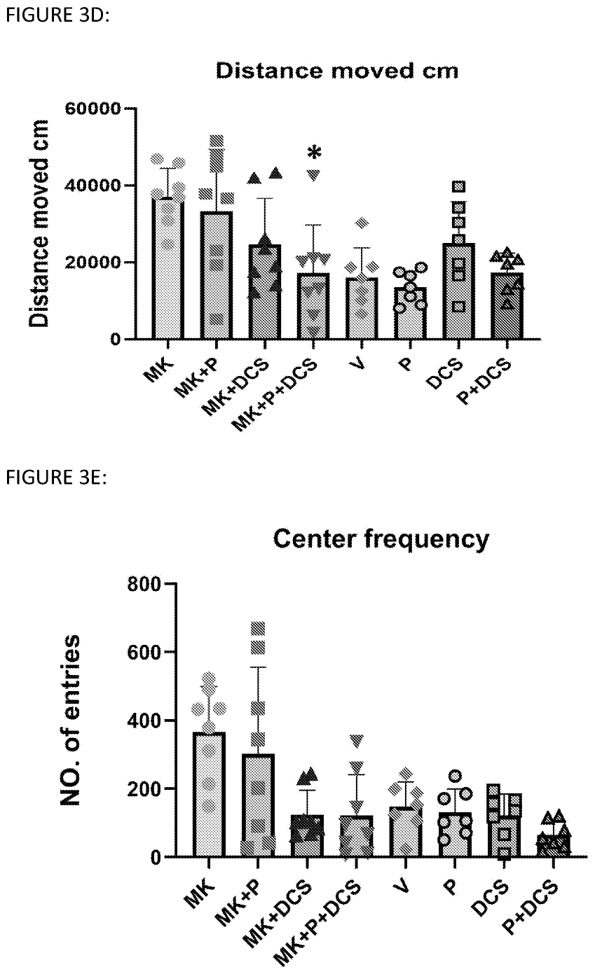

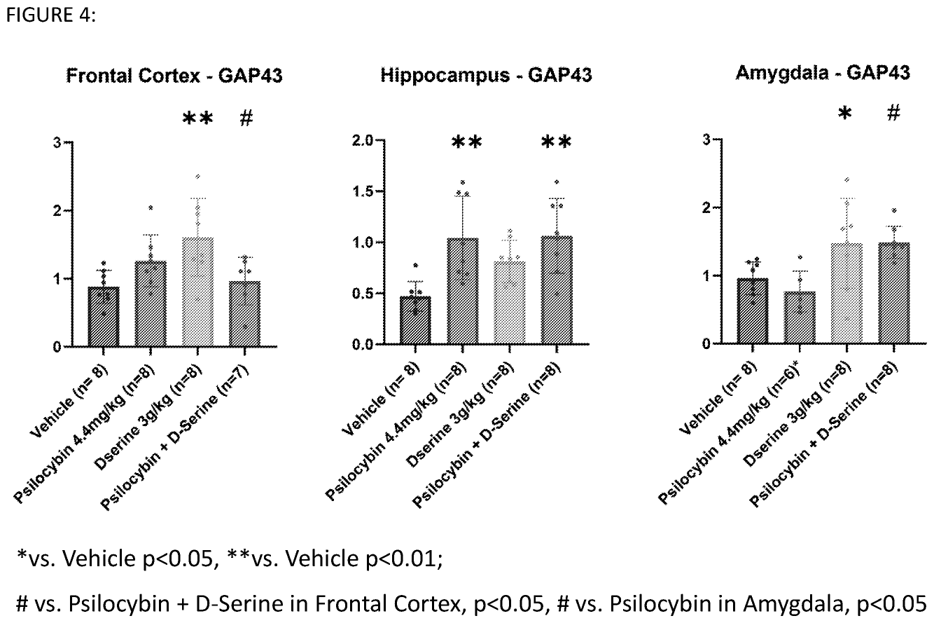

In some embodiments, the combinations/compositions/kits/methods/uses contemplate the combined use of a psychedelic drug and an antipsychotic possessing 5-HT2A antagonist properties, may follow an administration schedule such as that described in FIGS. 3 and 4, herein, or any similar appropriate staggered regimen as herein described.

In some embodiments, a method of treatment comprises the administration of a therapeutically effective amount of psilocybin, prodrug of psilocin, an active metabolite of psilocybin, or a prodrug of an active metabolite of psilocin to a subject in need thereof as described herein. In some embodiments, a method of treatment comprises the administration of a therapeutically effective amount of psilocybin as described herein.

In some embodiments, a method of treatment comprises the administration of a therapeutically effective amount of psilocin as described herein.

Some embodiments comprise psilocybin, a prodrug of psilocin, an active metabolite of psilocybin, or a prodrug of an active metabolite of psilocin for use in the treatment of an indication as described herein.

Some embodiments comprise psilocybin for use in the treatment of an indication as described herein. Some embodiments comprise psilocin for use in the treatment of an indication as described herein.

Some embodiments comprise the use of psilocybin, psilocin, a prodrug of psilocin, an active metabolite of psilocybin, or a prodrug of an active metabolite of psilocin in the manufacture of a medicament for the treatment of an indication as described herein.

In some aspects, the reference to psilocybin herein shall also be understood to encompass polymorphs and hydrates of psilocybin, along with the preparation and formulations thereof as disclosed in U.S. Application No. US2019/0119310 A1, which is incorporated by reference herein in its entirety.

In some embodiments, this invention provides a combination therapy comprising a psychedelic drug and an antipsychotic possessing 5-HT2A antagonist properties and an NMDA receptor agonist or partial agonist.

According to this aspect, and in some aspects, the combination therapy May include co-administration or proximal administration of three separate compositions, containing the psychedelic in a first composition, the antipsychotic possessing 5-HT2A antagonist properties in a second composition and an NMDA receptor agonist or partial agonist in a third composition. In some aspects, the combination therapy may include co-administration or proximal administration of two separate compositions, where a first composition comprises e.g. the psychedelic and the antipsychotic possessing 5-HT2A antagonist properties, or the psychedelic and NMDA receptor agonist or partial agonist, or the NMDA receptor agonist or partial agonist and antipsychotic possessing 5-HT2A antagonist properties, and the second composition comprising the NMDA receptor agonist or partial agonist, or antipsychotic possessing 5-HT2A antagonist properties, or the psychedelic, respectively, i.e. the second composition comprising the “third” agent not provided as part of the combination product of the first composition. In still a further embodied aspect, the invention contemplates a single composition comprising all 3 actives therein.

In some embodiments, the psychedelic drug is psilocybin, Dimethyltryptamine (DMT), 5-Methoxy-N, N-dimethyltryptamine (5-MeO-DMT), Mescaline, 3,4-methylenedioxymethamphetamine (MDMA) or Lysergic acid diethylamide (LSD) or a combination thereof.

In some embodiments, the antipsychotic possessing 5-HT2A antagonist properties is clozapine, olanzapine, quetiapine, risperidone, aripiprazole, asenapine, pizotifen, ziprasidone, chloropromazine, thioridazine, paloperidone, iloperidone, lurasidone, chlorprothixene, sertindole, lumateperone, zuclopenthixol, loxapine, amisulpiride, thiothixene, zotepine, cariprazine or a combination thereof.

In some embodiments, the antipsychotic possessing 5-HT2A antagonist properties is clozapine.

In some embodiments, the NMDA receptor agonist is at least a partial agonist.

In some embodiments, the NMDA receptor agonist is D-serine.

In some embodiments, the NMDA receptor agonist is D-cycloserine

In some embodiments, the invention provides a kit comprising the combination therapy as herein described.

In some embodiments, the invention provides a composition comprising the combination therapy as herein described.

In some embodiments, the invention provides a method of treating, reducing the incidence of, reducing the severity of or improving a pathology of a subject with schizophrenia, the method comprising administering to said subject a combination of a psychedelic drug and an antipsychotic possessing 5-HT2A antagonist properties.

In some embodiments, treating, reducing the incidence of, reducing the severity of or improving a pathology of a subject with schizophrenia comprises reducing or abrogating negative symptoms of schizophrenia.

According to this aspect and in some embodiments, the psychedelic drug and antipsychotic possessing 5-HT2A antagonist properties are administered simultaneously.

According to this aspect and in some embodiments, the psychedelic drug and antipsychotic possessing 5-HT2A antagonist properties are administered separately.

According to this aspect and in some embodiments, the combination of psychedelic drug and antipsychotic possessing 5-HT2A antagonist properties are provided in a kit of parts.

According to this aspect and in some embodiments, the psychedelic drug and antipsychotic possessing 5-HT2A antagonist properties are provided in a single composition.

Further according to this aspect, the invention contemplates combination therapy with psilocybin and clozapine. According to this aspect and in some embodiments, the invention contemplates administration of a single composition comprising psilocybin and clozapine, or in some embodiments, consisting essentially of psilocybin and clozapine as the active ingredients therein, but further containing excipients, etc.

Further according to this aspect, the invention contemplates combination therapy with psilocybin and D-serine. According to this aspect and in some embodiments, the invention contemplates administration of a single composition comprising psilocybin and D-serine, or in some embodiments, consisting essentially of psilocybin and D-serine as the active ingredients therein, but further containing excipients, etc.

Further according to this aspect, the invention contemplates combination therapy with psilocybin and D-cycloserine. According to this aspect and in some embodiments, the invention contemplates administration of a single composition comprising psilocybin and D-cycloserine, or in some embodiments, consisting essentially of psilocybin and D-cycloserine as the active ingredients therein, but further containing excipients, etc.

In other embodiments, this invention provides a method of treating, reducing the incidence of, reducing the severity of or improving a pathology of a subject with schizophrenia, the method comprising administering to said subject a combination of a psychedelic drug and an NMDA receptor agonist.

According to this aspect and in some embodiments, the NMDA receptor agonist is at least a partial agonist.

According to this aspect and in some embodiments, the NMDA receptor agonist is D-serine.

According to this aspect and in some embodiments, the NMDA receptor agonist is D-cycloserine

According to this aspect and in some embodiments, the treating, reducing the incidence of, reducing the severity of or improving a pathology of a subject with schizophrenia comprises reducing or abrogating negative symptoms of schizophrenia.

According to this aspect and in some embodiments, the NMDA receptor agonist enhances neuroplasticity in said subject beyond that achieved with administration of said psychedelic drug alone.

In some aspects, neuroplastic effects may be verified by any means known in the art, for example, as described in WIPO Patent Application Publication Number WO/2021/074448, fully incorporated by reference herein.

According to this aspect and in some embodiments, the treating, reducing the incidence of, reducing the severity of or improving a pathology of a subject with schizophrenia comprises further administering an antipsychotic possessing 5-HT2A antagonist properties.

According to this aspect and in some embodiments, the psychedelic drug and NMDA receptor agonist and optionally said antipsychotic possessing 5-HT2A antagonist properties are administered simultaneously.

According to this aspect and in some embodiments, the psychedelic drug and NMDA receptor agonist and optionally said antipsychotic possessing 5-HT2A antagonist properties are administered separately.

According to this aspect and in some embodiments, the combination of said psychedelic drug and NMDA receptor agonist and optionally the antipsychotic possessing 5-HT2A antagonist properties are provided in a kit of parts.

According to this aspect and in some embodiments, the combination of psychedelic drug and NMDA receptor agonist and optionally the antipsychotic possessing 5-HT2A antagonist properties are provided in a single composition.

In other embodiments, this invention provides a method of treating, reducing the incidence of, reducing the severity of or improving a pathology of a subject with depression, the method comprising administering to said subject a combination of a psychedelic drug and an NMDA receptor agonist or an antipsychotic possessing 5-HT2A antagonist properties and an NMDA receptor agonist.

According to this aspect and in some embodiments, the method further comprises administering to the subject an antipsychotic possessing 5-HT2A antagonist properties.

According to this aspect and in some embodiments, the treating, reducing the incidence of, reducing the severity of or improving a pathology of a subject with depression comprises treatment-resistant depression.

According to this aspect and in some embodiments, the psychedelic drug and antipsychotic possessing 5-HT2A antagonist properties are administered simultaneously and the NMDA receptor agonist is administered separately.

According to this aspect and in some embodiments, the psychedelic drug and antipsychotic possessing 5-HT2A antagonist properties are administered separately.

According to this aspect and in some embodiments, the psychedelic drug and antipsychotic possessing 5-HT2A antagonist properties are provided in a single composition.

According to this aspect and in some embodiments, the psychedelic drug and NMDA receptor agonist and optionally the antipsychotic possessing 5-HT2A antagonist properties are administered simultaneously.

According to this aspect and in some embodiments, the psychedelic drug and NMDA receptor agonist and optionally the antipsychotic possessing 5-HT2A antagonist properties are administered separately.

According to this aspect and in some embodiments, the combination of psychedelic drug and NMDA receptor agonist and optionally the antipsychotic possessing 5-HT2A antagonist properties are provided in a kit of parts.

According to this aspect and in some embodiments, the combination of psychedelic drug and NMDA receptor agonist and optionally the antipsychotic possessing 5-HT2A antagonist properties are provided in a single composition.

This invention also provides, in another embodiment, a method of treating, reducing the incidence of, reducing the severity of or improving a pathology of a subject with post-traumatic stress disorder, the method comprising administering to said subject a combination of a psychedelic drug and an antipsychotic possessing 5-HT2A antagonist properties and an NMDA receptor agonist.

According to this aspect and in some embodiments, the psychedelic drug and antipsychotic possessing 5-HT2A antagonist properties are administered simultaneously, and the NMDA receptor agonist is administered separately.

According to this aspect and in some embodiments, the psychedelic drug and said antipsychotic possessing 5-HT2A antagonist properties are administered separately.

According to this aspect and in some embodiments, the psychedelic drug and said antipsychotic possessing 5-HT2A antagonist properties are provided in a single composition.

According to this aspect and in some embodiments, the psychedelic drug and the NMDA receptor agonist and optionally the antipsychotic possessing 5-HT2A antagonist properties are administered simultaneously.

According to this aspect and in some embodiments, the psychedelic drug and NMDA receptor agonist and optionally the antipsychotic possessing 5-HT2A antagonist properties are administered separately.

According to this aspect and in some embodiments, the combination of psychedelic drug and NMDA receptor agonist and optionally the antipsychotic possessing 5-HT2A antagonist properties are provided in a kit of parts.

According to this aspect and in some embodiments, the combination of psychedelic drug and NMDA receptor agonist and optionally the antipsychotic possessing 5-HT2A antagonist properties are provided in a single composition.

This invention still further provides, in some embodiments, a method of enhancing neuroplasticity in a subject with schizophrenia, depression or post-traumatic stress disorder, the method comprising administering to said subject a combination of a psychedelic drug and an NMDA receptor agonist.

According to this aspect and in some embodiments, the psychedelic drug and NMDA receptor agonist are administered simultaneously.

According to this aspect and in some embodiments, the psychedelic drug and NMDA receptor agonist are administered separately.

According to this aspect and in some embodiments, the psychedelic drug and NMDA receptor agonist are provided in a single composition.

According to this aspect and in some embodiments, the combination of psychedelic drug and NMDA receptor agonist and optionally the antipsychotic possessing 5-HT2A antagonist properties are provided in a kit of parts.

In some embodiments, the combinations/compositions/kits/methods/uses are contemplated for applications in treating one or more neurocognitive disorders in a subject in need thereof, the method comprising administering to the subject a therapeutically effective amount of an NMDAR agonist or partial agonist, including a psychedelic drug, such as psilocybin and optionally an antipsychotic possessing 5-HT2A antagonist properties.

In other embodiments, there is provided herein a method for treating Parkinsonian syndrome or symptoms thereof in a subject in need thereof, comprising administering to the subject a therapeutically effective amount of an NMDAR agonist or partial agonist, including a psychedelic drug, such as psilocybin.

In some embodiments, the methods of this invention and the combinations/compositions/kits/methods/uses are contemplated for applications in treating one or more motor disorders, including for example, cerebral palsy, muscular dystrophy, spina bifida, spinal muscle atrophy (SMA), Parkinson's disease, amyotrophic lateral sclerosis (ALS), Ataxia, cervical dystonia, general dystonia, chorea, functional movement disorder, Huntington's disease, multiple system atrophy, myoclonus, Parkinsonism, Stroke or repeated head trauma, progressive supranuclear palsy, restless legs syndrome, tardive dyskinesia, Tourette syndrome, tremor, or Wilson's disease.

In some embodiments, the combinations/compositions/kits/methods/uses are contemplated for applications in treating attention-deficit hyperactivity disorder (ADHD) in a subject in need thereof, comprising administering to the subject/providing a therapeutically effective amount of an NMDAR agonist or partial agonist, including a psychedelic drug, such as psilocybin and optionally an antipsychotic possessing 5-HT2A antagonist properties.

In some embodiments, the combinations/compositions/kits/methods/uses are contemplated for applications in treating epilepsy in a subject in need thereof, comprising administering to the subject/providing a therapeutically effective amount of an NMDAR agonist or partial agonist, including a psychedelic drug, such as psilocybin and optionally, an antipsychotic possessing 5-HT2A antagonist properties.

In some embodiments, the combinations/compositions/kits/methods/uses are contemplated for applications in treating an autism spectrum disorder (ASD) or a symptom thereof in a subject in need thereof, comprising administering to the subject/providing a therapeutically effective amount of an NMDAR agonist or partial agonist, including a psychedelic drug, such as psilocybin and/or an antipsychotic possessing 5-HT2A antagonist properties.

In some embodiments, the combinations/compositions/kits/methods/uses are contemplated for applications in treating one or more sleep-wake disorders in a subject in need thereof, comprising administering to the subject/providing a therapeutically effective amount of an NMDAR agonist or partial agonist, including a psychedelic drug, such as psilocybin and/or an antipsychotic possessing 5-HT2A antagonist properties.

In some embodiments, the combinations/compositions/kits/methods/uses are contemplated for applications in treating chronic pain in a subject in need thereof, comprising administering to the subject/providing a therapeutically effective amount of an NMDAR agonist or partial agonist, including a psychedelic drug, such as psilocybin and/or an antipsychotic possessing 5-HT2A antagonist properties.

In some embodiments, the combinations/compositions/kits/methods/uses are contemplated for applications in reducing inflammation in a subject in need thereof, comprising administering to the subject/providing a therapeutically effective amount of a psychedelic drug, such as psilocybin and an NMDAR agonist or partial agonist; or a psychedelic drug, such as clozapine CLZ (or another 5HT2 antipsychotic).

In some embodiments, the combinations/compositions/kits/methods/uses are contemplated for applications in treating stroke in a subject in need thereof, comprising administering to the subject/providing a therapeutically effective amount of an NMDAR agonist or partial agonist, including a psychedelic drug, such as psilocybin and/or an antipsychotic possessing 5-HT2A antagonist properties.

In some embodiments, the combinations/compositions/kits/methods/uses are contemplated for applications in treating amyotrophic lateral sclerosis (ALS) in a subject in need thereof, comprising administering to the subject/providing a therapeutically effective amount of an NMDAR agonist or partial agonist, including a psychedelic drug, such as psilocybin and/or an antipsychotic possessing 5-HT2A antagonist properties.

In some embodiments, the combinations/compositions/kits/methods/uses are contemplated for applications in treating neuropsychiatric disorders (such as schizophrenia or OCD, or generalized anxiety disorder, or panic disorder) or neurocognitive disorders (e.g., Alzheimer's Disease/Parkinson's Disease) in a subject.

According to these aspects and in some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties.

According to this aspect and embodiment, the in some combinations/compositions/kits/methods/uses cause a demonstrated improvement in one or more of the following: the Mini-Mental State Exam (MMSE), the Mini-Cog test, a CANTAB test, a Cognigram test, a Cognivue test, a Cognition test, or an Automated Neuropsychological Assessment Metrics test.

According to this aspect and in some embodiment, the combinations/compositions/kits/methods/uses provide for the inclusion of one or more additional therapeutics administered in combination with the combinations described.

For example, the one or more additional therapeutics may be an antidepressant, cholinesterase inhibitors, AChE (acetylcholinesterase) inhibitor, BChE (Butyrylcholinesterase) inhibitor, NMDA (N-methyl-D-aspartate) antagonist, or combinations thereof. A non-limiting list of exemplary types of antidepressants includes: SSRIs (selective serotonin reuptake inhibitors), MAOIs (monoamine oxidase inhibitors), SNRIs (serotonin and norepinephrine reuptake inhibitors), and TCAs (tricyclic antidepressants). For example, the antidepressant may be citalopram, escitalopram, fluoxetine, fluvoxamine, paroxetine, sertraline, vortioxetine, vilazodone, duloxetine, venlafaxine, desvenlafaxine, levomilnacipran, amitriptyline, amoxapine, clomipramine, desipramine, desipramine, doxepin, imipramine, nortriptyline, protriptyline, trimipramine, mirtazapine, bupropion, trazodone, vortioxetine, or vilazodone.

In some embodiments, the combinations/compositions/kits/methods/uses are contemplated wherein same are provided to a subject in need thereof, for example, in cases of depression, including treatment resistant depression, or in some aspects, with any of the other neuropsychiatric disorders as described herein, such as, for example, panic disorder, OCD, anxiety disorder, to further include treatment with the combinations/compositions/kits as herein described, prior to, proximal to, or coincident with adjunct psychotherapy.

In some embodiments, the term “neurocognitive disorder” refers to a wide range of disorders that affect the brain, and are often associated with decreased or altered mental function.

In some embodiments, the neurocognitive disorder is a major neurocognitive disorder. In some embodiments, the neurocognitive disorder is mild neurocognitive disorder.

In some embodiments, the neurocognitive disorder is dementia. In some embodiments, the dementia is late onset dementia (with or without hallucinations), hallucinations co-occurrent and due to late onset dementia; mild dementia; mixed dementia; moderate dementia; organic dementia; presbyophenia; presbyophrenic psychosis; presenile dementia; presenile dementia with delirium; presenile dementia with depression, presenile dementia with delusions; primary degenerative dementia; senile dementia; senile dementia with delusions; senile dementia with delirium, depression, paranoia, or psychosis; or severe dementia.

In some embodiments, the neurocognitive disorder is caused by traumatic brain injury, such as bleeding into the brain (intracerebral hemorrhage), bleeding into the space around the brain (subarachnoid hemorrhage), blood clot inside the skull causing pressure on brain (subdural or epidural hematoma), or concussion.

In some embodiments, the neurocognitive disorder is caused by a breathing condition, such as low oxygen in the body (hypoxia) or high carbon dioxide level in the body (hypercapnia).

In some embodiments, the neurocognitive disorder is caused by a cardiovascular disorder, such as dementia due to many strokes (multi-infarct dementia), heart infections (endocarditis, myocarditis), stroke, or transient ischemic attack (TIA).

In some embodiments, the neurocognitive disorder is caused by a degenerative disorder, such as Alzheimer's disease (also called senile dementia, Alzheimer type), Creutzfeldt-Jakob disease, Diffuse Lewy body disease, Huntington's disease, Multiple sclerosis, Normal pressure hydrocephalus, Parkinson's disease, or Pick disease. In some embodiments, the neurocognitive disorder is due to one or more of Alzheimer's disease, Lewy Body Dementia, Traumatic Brain Injury, Prion Disease, HIV Infection, Parkinson's disease, or Huntington's disease.

In some embodiments, the neurocognitive disorder is dementia due to metabolic causes, such as kidney disease, liver disease, thyroid disease (hyperthyroidism or hypothyroidism), or vitamin deficiency (B1, B12, or folate).

In some embodiments, the neurocognitive disorder is caused by a drug or alcohol-related condition, such as alcohol withdrawal state, intoxication from drug or alcohol use, Wernicke-Korsakoff syndrome (a long-term effect of excessive alcohol consumption or malnutrition), or withdrawal from drugs (such as sedative-hypnotics and corticosteroids).

In some embodiments, the neurocognitive disorder is caused by an infection, such has any sudden onset (acute) or long-term (chronic) infection. For example, the infection may be blood poisoning (septicemia), brain infection (encephalitis), meningitis (infection of the lining of the brain and spinal cord), prion infections (e.g., mad cow disease), or late-stage syphilis.

In some embodiments, the neurocognitive disorder is caused by complications from cancer and/or cancer treatment with chemotherapy.

In some embodiments, the neurocognitive disorder is caused by depression, neurosis, or psychosis.

In some embodiments, the neurocognitive disorder is Mild Cognitive Impairment.

In some embodiments, the subject has one or more diseases, disorders, or conditions that are comorbid with the neurocognitive disorder. For example, the one or more comorbidities may be hypertension, connective tissue disease, depression, diabetes, or chronic pulmonary disease.

In some embodiments, the neurocognitive disorder is due to Alzheimer's disease (AD), such as sporadic Alzheimer's Disease or Familial Alzheimer's Disease.

According to this aspect and in some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating a Parkinsonian syndrome or symptom thereof in a subject in need thereof. In some embodiments, the Parkinsonian syndrome is Parkinson's disease. In some embodiments, the Parkinsonian syndrome is drug-induced.

In some embodiments, the Parkinsonian syndrome is an atypical Parkinsonian disorder. In some embodiments, the atypical parkinsonian disorder is multiple system atrophy progressive supranuclear palsy, corticobasal degeneration, or dementia with Lewy bodies.

In some embodiments, the subject suffers from a motor symptom or a nonmotor symptom, or combinations thereof. In some embodiments, the motor symptom is bradykinesia, rigidity, tremor, rest tremor, postural instability, stiffness, slowness, imbalance, or combinations thereof. In some embodiments, the nonmotor symptom is cognitive impairment, olfactory loss, sleep dysfunction, autonomic dysfunction, psychiatric disturbance, fatigue, softening of the voice, sialorrhea, trouble swallowing, or combinations thereof.

In some embodiments, the subject has one or more diseases, disorders, or conditions that are comorbid with a Parkinsonian syndrome. In some embodiments, the comorbidity results from a symptom of a Parkinsonian syndrome. In some embodiments, the comorbidity is selected from a neuropsychiatric disturbance, a sleep disorder, melanoma, neurogenic orthostatic hypotension, pseudobulbar affect, anemia, hypertension, type 2 diabetes, restless leg syndrome, cancer, or combinations thereof. In some embodiments, the comorbidity is a neuropsychiatric disturbance, and wherein the neuropsychiatric disturbance is dementia, depression, psychosis, apathy, anxiety, hallucinations, or combinations thereof. In some embodiments, comorbidity is a sleep disorder (e.g., rapid eye movement sleep behavior disorder), and wherein the sleep disorder is daytime drowsiness and sleepiness, sleep attacks, insomnia, or rapid eye movement sleep behavior disorder.

According to this aspect, and in some embodiments, the invention contemplates, when treating a Parkinsonian syndrome or symptom thereof in a subject in need thereof further administering to the subject at least one additional therapy. In some embodiments, the additional therapy is exercise, physical, occupational, or speech therapy. In some embodiments, the additional therapy is a dopaminergic medication. In some embodiments, the additional therapy is carbidopa-levodopa, entacopone, tolcapone, carbidopa, levodopa entacapone, pramipexole, ropinirol, apomorphine, rotigotine, selegiline, rasagiline, safinamide, amantadine, istradefylline, trihexyphenidyl, benztropine/benzatropine, or combinations thereof.

In some embodiments, the methods for treating a Parkinsonian syndrome or symptom thereof described herein ameliorate the Parkinsonian syndrome, or at least one symptom thereof, in the subject. In some embodiments, one or more of the following scales are used to assess the efficacy of treating Parkinson's disease according to the methods of the disclosure: the Hochn and Yahr staging scale, the Unified Parkinson's Disease Rating Scale (UPDRS), the Clinical Impression of Severity Index (CISI-PD), the Movement Disorders Society Sponsored Revision of the Unified Parkinson's Disease Rating Scale (MDS-UPDRS), Scales for Outcomes in Parkinson's Disease-motor (SCOPA-Motor), the Schwab & England Activities of Daily Living Scales (SES), the Self-assessment Parkinson's Disease Disability Scale (SPDDS), the Postural Instability and Gait Difficulty score (PIGD), Freezing of Gait Questionnaire (FOGQ), the Nonmotor Symptoms Questionnaire (NMSQuest), the Nonmotor Symptoms Scale (NMSS), Unified Dyskinesia Rating Scale (UDysRS), the Wearing-off Questionnaires (WOQ), self-reported total sleep time on the Pittsburgh Sleep quality index, the Beck Depression inventory, the Insomnia Severity Index, or combinations thereof.

In some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having ADHD.

As used herein, “Attention-deficit hyperactivity disorder” (ADHD) is a mental disorder of the neurodevelopmental type characterized by one or more of inattention, hyperactivity, and impulsivity, which are otherwise not appropriate for a person's age. It is commonly diagnosed in childhood, and is one of the most frequent condition affecting school-aged children. In children, the primary symptoms of inattention, hyperactivity, and impulsivity can lead to disruptive behavior at home and in school, which is a typical precursor to clinical referral for diagnosis and treatment. Hyperactivity often decreases in adulthood, however inattention, disorganization, and impulsivity typically persist, causing functional challenges to the patient on a day-to-day basis.

In some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting at least one disease, disorder, or condition selected from oppositional defiant disorder, learning difficulties, depression, anxiety, bipolar disorder, substance use disorders, autism spectrum disorders, personality disorder, obsessive compulsive disorder, or combinations thereof.

In some embodiments, the subject is administered an additional therapy in addition to the combinations/compositions as herein described. In some embodiments, the additional therapy is a stimulant, a norepinephrine reuptake inhibitor, an a-adrenergic agonist, a tricyclic antidepressant, modafinil, or combinations thereof. In some embodiments, the additional therapy is a stimulant (e.g., an amphetamine or methylphenidate). In some embodiments, the additional therapy is a norepinephrine reuptake inhibitor (e.g., atomoxetine or reboxetine).

In some embodiments, administration of the NMDAR agonist or partial agonist and psilocybin (or active metabolite thereof) or a psychedelic drug as herein described to a subject alleviates at least one sign or symptom of ADHD.

In some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having Epilepsy.

In some embodiments, the epilepsy is generalized epilepsy, epilepsy with myoclonic absence seizures, focal epilepsy, generalized and focal epilepsy, unknown if generalized or focal epilepsy, autosomal dominant nocturnal frontal lobe epilepsy, childhood absence epilepsy, benign rolandic epilepsy, Doose syndrome, Dravet syndrome, early myoclonic encephalopathy, Jeavons syndrome, epilepsy in infancy with migrating focal seizures, epileptic encephalopathy with continuous spike and wave during sleep, febrile illness-related epilepsy syndrome, frontal lobe epilepsy, west syndrome, juvenile absence epilepsy, juvenile myoclonic epilepsy, Landau-Kleffner syndrome, Lennox-Gastaut syndrome, Ohtahara syndrome, Panayiotopoulos syndrome, progressive myoclonic epilepsy, reflex epilepsy, or temporal lobe epilepsy. In some embodiments, the subject in need thereof has generalized tonic-clonic, convulsive, absence, myoclonic, clonic, tonic, or atonic seizures.

Some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having bipolar disorder, ADHD, depression, anxiety, or combinations thereof. In some embodiments, the subject being treated suffers from migraine, cognitive impairment, stroke, cerebrovascular disease, or combinations thereof.

According to this aspect and in some embodiments, the invention contemplates the combination/compositions/methods/uses/kits providing for the administration to the subject of an additional therapy. In some embodiments, the additional therapy is a sodium channel blocker, calcium current inhibitor, gamma-aminobutyric (GABA) enhancer, glutamate receptor antagonists, carbonic anhydrase inhibitor, hormone, an N-methyl-D-aspartate (NMDA) receptor antagonist, synaptic vesicle glycoprotein 2A (SV2A) ligand, α-amino-3-hydroxy-5-methyl-4-isoxazolepropionic acid (AMPA)/Kainate receptor antagonist, or combinations thereof. In some embodiments, the additional therapy is a sodium channel blocker, and the sodium channel blocker is phenytoin, fosphenytoin, carbamazepine, lamotrigine, or valproate. In some embodiments, the additional therapy is a calcium channel antagonist, and wherein the calcium current inhibitor is ethosuximide or valproate. In some embodiments, the additional therapy is a GABA enhancer, and wherein the GABA enhancer is a benzodiazepine, barbiturate, progabide, progesterone, ganaxolone, vigabatrin, tiagabine, gabapentin, or valproate. In some embodiments, the additional therapy is an NMDA receptor antagonist, and the NMDA receptor antagonist is felbamate or levetiracetam. In some embodiments, the additional therapy is an AMPA/Kainate receptor antagonist, and wherein the AMPA/Kainate receptor antagonist is topiramate.

In some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having Autism.

Autism spectrum disorder (ASD) is a neurodevelopmental syndrome characterized by core deficits in social interaction and communication, presence of repetitive and restricted patterns of behavior and interests, and/or unusual reactivity to sensory input.

In some embodiments, the ASD is autistic disorder, Asperger's syndrome, pervasive developmental disorder not otherwise specified (PDD-NOS), childhood disintegrative disorder, or combinations thereof. In some embodiments, the sign or symptom of ASD is irritability, repetitive behavior, restricted behaviors, unusual reactivity to sensory stimuli, social communication deficits, aggression, self-injurious behavior, motor impairment, cognitive deficits, or combinations thereof.

In some embodiments, the subject suffers from cognitive deficits in cognitive flexibility, sustained attention, working memory, episodic memory, executive function, or combinations thereof.

In some embodiments, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having anxiety disorders, sleep-wake disorder, impulse-control, disruptive behavior, conduct disorder, depressive disorders, obsessive-compulsive and related disorders, bipolar disorder, schizophrenia, or combinations thereof. In some embodiments, the comorbidity is an inflammatory disorder, gastrointestinal disorder, epilepsy, or a combination thereof.

In some embodiments, according to these aspect, the invention contemplates further administering to the subject one additional therapeutic agent. In some embodiments, the least one additional therapeutic agent is risperidione or aripiprazole. In some embodiments, the at least one additional therapeutic agent is an antidepressant, such as SSRIs (selective serotonin reuptake inhibitors), MAOIs (monoamine oxidase inhibitors), SNRIs (serotonin and norepinephrine reuptake inhibitors), and TCAs (tricyclic antidepressants). For example, the antidepressant may be citalopram, escitalopram, fluoxetine, fluvoxamine, paroxetine, sertraline, vortioxetine, vilazodone, duloxetine, venlafaxine, desvenlafaxine, levomilnacipran, amitriptyline, amoxapine, clomipramine, desipramine, desipramine, doxepin, imipramine, nortriptyline, protriptyline, trimipramine, mirtazapine, bupropion, trazodone, vortioxetine, or vilazodone,

In some embodiments, the Autism Diagnostic Observation Schedule, Second Edition (ADOS-2), Autism Diagnostic Interview-Revised (ADI-R), Childhood Autism Rating Scale, Second Edition (CARS2), Vineland-II Adaptive Behavior Scales (VABS-2), Aberrant Behavior Checklist (ABC), Child Behavior Checklist (CBCL), Autism Behavior Inventory (ABI), Social Responsiveness Scale, Second Edition (SRS-2), Repetitive Behavior Scale-Revised (RBS-R), the Ohio Autism Clinical Impressions Scale-Improvement (OACIS-I), Ohio Autism Clinical Impressions Scale-Severity (OACIS-S), the Gilliam Autism Rating Scale-Third Edition (GARS-3), Social Communication Questionnaire (SCQ), Autism Spectrum Quotient (AQ), Adult Repetitive Behavior Questionnaire-2 (RBQ-2A), or combinations thereof, are used to assess the efficacy of treating according to the methods of the disclosure.

In some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having Sleep-Wake Disorders.

Sleep-wake disorders are a class of a diseases or disorders including insomnia disorder, hypersomnolence disorder, narcolepsy, breathing-related sleep disorders (such as central sleep apnea), circadian rhythm sleep-wake disorders, non-rapid eye movement sleep arousal disorders, nightmare disorder, rapid eye movement sleep behavior disorder, restless leg syndrome, and substance/medication-induced sleep disorder. Individuals with these disorders typically present with sleep-wake complaints of dissatisfaction regarding the quality, timing, and amount of sleep, which often results in daytime distress.

As used herein, the term insomnia refers to an individual's difficulty with sleep. It is diagnosed using the following criteria: (1) difficulty falling asleep, staying asleep or nonrestorative sleep; (2) this difficulty is present despite adequate opportunity and circumstance to sleep; (3) this impairment in sleep is associated with daytime impairment or distress; and (4) this sleep difficulty occurs at least 3 times per week and has been a problem for at least 1 month. Insomnia disorder can be classified as chronic (sleep disturbances occur at least three times a week and have been present for the last 3 months), short-term (sleep disturbances have been present for over a period of up to 3 months) and other (difficulty in initiating or maintaining sleep that does not meet the criteria of chronic insomnia or short-term insomnia disorder). Primary insomnia occurs independently of other factors and may be related to a general psychophysiological hyperarousal.

In some embodiments, a method of treating one or more sleep-wake disorders in a subject in need thereof comprises administering to the subject an effective amount of an NMDAR agonist or partial agonist, further in combination with a psychedelic drug and/or an antipsychotic possessing 5-HT2A antagonist properties. In some embodiments, the sleep-wake disorder is insomnia, hypersomnolence, narcolepsy, cataplexy, idiopathic hypersomnia, sleep paralysis, hypnagogic hallucinations, hypnopompic hallucinations, a breathing-related sleep disorder, a circadian rhythm sleep-wake disorder, a non-24 hour sleep wake disorder, a non-rapid eye movement sleep arousal disorder, a nightmare disorder, a rapid eye movement sleep behavior disorder, restless leg syndrome, a medication-induced sleep disorder, or a substance-induced sleep disorder.

In some embodiments, the sleep-wake disorder is insomnia. In some embodiments, the insomnia is chronic. In some embodiments, the insomnia is short term.

In some embodiments, the sleep-wake disorder is hypersomnolence. In some embodiments, the hypersomnolence is characterized by one or more of excessive daytime sleepiness, excessive daytime somnolence, and/or hypersomnia.

In some embodiments, the sleep wake disorder is narcolepsy, such as type 1 or type 2 narcolepsy.

In some embodiments, the subject has excessive daytime sleepiness, cataplexy, sleep paralysis, hypnagogic hallucinations, hypnopompic hallucinations, or combinations thereof prior to treatment with psilocybin or an active metabolite thereof. In some embodiments, the subject experiences an improvement in excessive daytime sleepiness, cataplexy, sleep paralysis, hypnagogic hallucinations, hypnopompic hallucinations or combinations thereof during treatment with psilocybin or an active metabolite thereof

In some embodiments, the subject experiences an improvement in excessive daytime sleepiness, cataplexy, sleep paralysis, hypnagogic hallucinations, hypnopompic hallucinations or combinations thereof after treatment with psilocybin or an active metabolite thereof

In some embodiments, the sleep-wake disorder is one or more breathing-related sleep disorders. For example, the breathing-related sleep disorder may be chronic snoring, upper airway resistance syndrome, sleep apnea, or obesity hypoventilation syndrome. In some embodiments, the breathing-related sleep disorder is sleep apnea, such as central sleep apnea (CSA). In some embodiments, the central sleep apnea is primary CSA, Cheyne-Stokes Breathing (CSB), high-altitude periodic breathing, CSA due to a medical condition without CSB, central sleep apnea due to a medication or substance, Treatment Emergent Central Apnea, or a combination thereof. In some embodiments, the subject experiences 1-30 fewer sleep apneas per hour of sleep after treatment with psilocybin. For example, the subject may experience a reduction in sleep apneas per hour of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more.

In some embodiments, the subject shows improvement in one or more of the following after treatment with psilocybin: mean sleep latency (MSL); multiple sleep latency test (MSLT); hypocretin (orexin) levels; sleep onset rapid eye movement periods (SOREMPs) in Epworth Sleepiness Scale (ESS); Maintenance of Wakefulness Test (MVVT) scores; cataplexy and cataplexy-like episodes; objective and subjective sleep latency; Total Sleep Time (TST); polysomnography; insomnia severity index (ISI) questionnaire; narcolepsy severity scale; Pittsburgh Sleep Quality Index score; Epworth Sleepiness Scale; Groningen Sleep Quality Questionnaire; Apnoea Hypopnea Index; and the Nightmare Experience Scale.

In some embodiments, the subject demonstrates an improvement in their MSLT after treatment with the combinations/compositions/kits as described herein as compared to their MSLT score prior to treatment. In some embodiments, the subject demonstrates an improvement of 1-10 minutes MSLT after treatment with psilocybin as described herein as compared to their MSLT score prior to treatment. In some embodiments, the subject demonstrates an improvement of 1-5 minutes MSLT after treatment with psilocybin as described herein as compared to their MSLT score prior to treatment. In some embodiments, the subject demonstrates an improvement of 1-3 minutes MSLT after treatment with psilocybin as described herein as compared to their MSLT score prior to treatment.

In some embodiments, the subject has one or more diseases, disorders, or conditions that are comorbid with the sleep-wake disorder. For example, the subject may have one or more of mood disorders, affective disorders, neurodegenerative disorders, neurodevelopmental disorders, autism spectrum disorders, and substance abuse disorders. In some embodiments, the subject has major depressive disorder, mania, depression, anxiety, psychosis, attention deficit hyperactivity disorder (ADHD), Parkinson's disorder, autism spectrum disorder (ASD), panic attacks, one or more social phobias, one or more eating disorders, and/or schizophrenia.

In some embodiments, the method of treating one or more sleep-wake disorders in a subject in need thereof further comprises administering to the subject at least one additional therapeutic agent. In some embodiments, the therapeutic agent increases serotonergic activity. In some embodiments, the therapeutic agent is a selective serotonin reuptake-inhibitor.

In some embodiments, the method of treating one or more sleep-wake disorders in a subject in need thereof further comprises administering to the subject cognitive behavioral therapy.

In some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having Pain, Including Chronic Pain.

As used herein, the term “chronic pain” refers to pain that lasts longer than the usual course of an acute injury or disease, such as pain that recurs for months or years. “Nociceptive pain” is a high-threshold pain activated in the presence of intense stimuli, such as touching something too hot, cold, or sharp. It minimizes contact with harmful stimuli and demands immediate action and attention. “Neuropathic pain” is a chronic pain caused by lesion or disease of the somatosensory system and can lead to altered transmission of sensory signals to the spinal cord and brain. Conditions associated with neuropathic pain include multiple sclerosis, diabetic neuropathy, post-herpetic neuralgia, brachial plexus injury, allodynia, human immunodeficiency virus (HIV) infection, amputation, nerve injury pain, stroke, cancer-related pain, trigeminal neuralgia, central neuropathic pain, post-traumatic neuropathy, postsurgical neuropathy, cervical and lumbar polyradiculopathies, leprosy, autoimmune disorders, inflammatory disorders, channelopathies and metabolic disorders. Additional examples of types of pain include visceral pain and bone pain.

In some embodiments, the method for treating a subject in need thereof further comprises administering to the subject at least one additional therapeutic. In some embodiments, the at least one additional therapeutic is a tricyclic antidepressant or a serotonin-noradrenaline reuptake inhibitor (SNRI). In some embodiments, the at least one additional therapeutic is pregabalin or gabapentin. In some embodiments, the at least one additional therapeutic is lidocaine, capsaicin, tramadol, botulinum toxin A, oxycodone, morphine, fentanyl, a cannabinoid, ketamine, acetaminophen, a nonsteroidial anti-inflammatory drug, an opioid, calcitonin,

In some embodiments, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having a stroke.

In some embodiments, the combinations/compositions/kits/methods/uses are contemplated wherein same are provided to a subject in need thereof to treat stroke, which in some embodiments, is an ischemic stroke, or in some embodiments, is a hemorrhagic stroke.

In some embodiments the combinations/compositions/kits/methods/uses as provided to a subject in need thereof improves a sign or symptom of stroke. The sign or symptom of stroke may be, for example, paralysis, numbness or weakness in the arm, face, or leg, trouble speaking or understanding speech, confusion, slurring speech, vision problems, trouble walking, loss of balance or coordination, dizziness, or headache.

In some embodiments, the sign or symptom of stroke is improved within 1 hour of administration. In some embodiments, the sign or symptom of stroke is improved within 12 hours of administration.

In some embodiments, the sign or symptom of stroke is improved for a period of at least 1 month after administration. In some embodiments, the sign or symptom of stroke is improved for a period of at least 3 months after administration. In some embodiments, the sign or symptom of stroke is improved for a period of at least 12 months after administration.

In some embodiments, the combinations/compositions/kits/methods/uses as provided to a subject in need thereof improves a sign or symptom of stroke stroke in a subject and further comprises administering a therapeutically effective amount of at least one additional therapeutic. The additional therapeutic drug may be, for example an anti-platelet drug (e.g., aspirin) or an anti-coagulant (e.g., warfarin, dabigatran, rivaroxaban, apizaban, edoxaban).

In some embodiments, the combinations/compositions/kits/methods/uses as provided to a subject in need thereof improves a condition caused by the stroke. In some embodiments, the condition caused by the stroke is paralysis, cognitive issues, difficulty understanding speech, difficulty speaking, difficulty controlling or expressing emptions, numbness, pain in the hands or feet, trouble chewing or swallowing, problems with bladder or bowel control.

In some embodiments, the condition caused by the stroke is improved within 24 hours of administration. In some embodiments, the condition caused by the stroke is improved within 1 week of administration.

In some embodiments, the condition caused by the stroke is improved for a period of at least 1 month after administration. In some embodiments, the condition caused by the stroke is improved for a period of at least 3 months after administration. In some embodiments, the condition caused by the stroke is improved for a period of at least 12 months after administration.

In other embodiments, the combinations/compositions/kits/methods/uses as provided to a subject in need thereof improves a sign or symptom of ALS. ALS is a progressive neurodegenerative disease. ALS is also known as Motor Neuron Disease (MND), Lou Gehrig's Disease, and Charcot's disease.

In some embodiments, administering the combinations/compositions/kits as herein described improves a sign or symptom of ALS. In some embodiments, the sign or symptom of ALS is muscle twitching, muscle weakness, muscle stiffness, difficulty speaking, difficulty swallowing, difficulty breathing, cognitive impairment, or pain.

In some embodiments, the sign or symptom of ALS is improved within 24 hours of administration. In some embodiments, the sign or symptom of ALS is improved within 1 week of administration.

In some embodiments, the sign or symptom of ALS is improved for a period of at least 1 month after administration. In some embodiments, the sign or symptom of ALS is improved for a period of at least 3 months after administration. In some embodiments, the sign or symptom of ALS is improved for a period of at least 12 months after administration.

In some embodiments, administering the combinations/compositions/kits as herein described improves a sign or symptom of Multiple sclerosis (MS). In some embodiments, the MS is clinically isolated syndrome (CIS). In some embodiments, the MS is relapsing-remitting MS (RRMS).

In some embodiments, the MS is primary progressive MS (PPMS). In some embodiments, the MS is secondary progressive MS (SPMS)

In some embodiments, administering the psilocybin improves a sign or symptom of MS. In some embodiments, the improved sign or symptom of MS can include a neurological symptom or sign, such as an autonomic, visual, motor, or sensory problem. In some embodiments, the improved sign or symptom of MS can include double vision, blindness in one eye, muscle weakness, trouble with sensation, trouble with coordination, loss of sensitivity, changes in sensation such as tingling, pins and needles or numbness, muscle weakness, blurred vision, very pronounced reflexes, muscle spasms, or difficulty in moving; difficulties with coordination and balance (ataxia); problems with speech or swallowing, visual problems (nystagmus, optic neuritis or double vision), feeling tired, acute or chronic pain, and bladder and bowel difficulties (such as neurogenic bladder). In some embodiments, the improved sign or symptom of MS can include difficulties thinking and emotional problems such as depression or unstable mood. In some embodiments, the improved sign or symptom of MS can include a reduction or decrease in Uhthoffs phenomenon, a worsening of symptoms due to exposure to higher than usual temperatures, and Lhermitte's sign, an electrical sensation that runs down the back when bending the neck. In some embodiments, after administration of psilocybin a subject demonstrates an improvement in their expanded disability status scale (EDSS) and/or multiple sclerosis functional composite score.

In some embodiments, the sign or symptom of MS is improved within 24 hours of administration. In some embodiments, the sign or symptom of MS is improved within 1 week of administration.

In some embodiments, the sign or symptom of MS is improved for a period of at least 1 month after administration. In some embodiments, the sign or symptom of MS is improved for a period of at least 3 months after administration. In some embodiments, the sign or symptom of MS is improved for a period of at least 12 months after administration.

In some embodiments, the method for treating MS a subject in need thereof further comprises administering to the subject at least one additional therapeutic to treat MS. In some embodiments, the at least one additional therapeutic is interferon beta-la, interferon beta-1b, glatiramer acetate, mitoxantrone, natalizumab, fingolimod, teriflunomide, dimethyl fumarate, alemtuzumab, ocrelizumab, siponimod, cladribine, and ozanimod.

In some embodiments, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having an obsessive-compulsive disorder (OCD).

In some embodiments, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having an anxiety disorder.

In some embodiments, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject requiring treatment of substance abuse.

In some embodiment, the combinations/compositions/kits/methods/uses are contemplated wherein same are provided to a subject in need thereof, wherein a therapeutically effective amount of an NMDAR agonist or partial agonist, is provided further in combination with a psychedelic drug, such as psilocybin and/or an antipsychotic possessing 5-HT2A antagonist properties for treating, ameliorating or otherwise positively affecting a subject having a disease or disorder as described in US2009203750-A1, AU-2005300045-A1, AU-2009214724-A1, US2019201402-A1, WO-2022067165-A1, U.S. Pat. No. 11,254,640-B2, EP-3519816-A1, WO-2021076572-A1 or WO-2020176599-A1, which are hereby incorporated by reference in their entirety.

It will appreciated herein be that the combinations/compositions/kits/methods/uses are contemplated wherein same are provided to a subject in need thereof, to address schizophrenia and numerous neuropsychiatric conditions, including all the embodied conditions described herein, where the combinations/compositions/kits/may be particularly helpful in treatment resistant conditions, such as, for example, treatment resistant depression, and others as will be appreciated by the skilled artisan. According to this aspect and in some embodiments, “treatment-resistant” conditions refers to a failure to respond to known treatments when provided at sufficient dosage for a sufficient time to typically elicit a therapeutic effect in other subjects with the same condition.

In various embodiments, a synergistic effect is observed when the combinations/compositions/kits as described herein are administered. A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination.

The regimen of administration may affect what constitutes an effective amount. The therapeutic formulations may be administered to the subject either prior to or after the onset of a neurological disease or disorder or a disease or disorder that is affected by, associated with, or would benefit from the psychedelic drug and an antipsychotic possessing 5-HT2A antagonist properties combination; psychedelic drug and an NMDA receptor agonist or partial agonist combination; antipsychotic possessing 5-HT2A antagonist properties and an NMDA receptor agonist or partial agonist combination; or psychedelic drug and an antipsychotic possessing 5-HT2A antagonist properties and an NMDA receptor agonist or partial agonist combination.

Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

In some aspects, the invention specifically contemplates combination therapy comprising a psychedelic drug and an antipsychotic possessing 5-HT2A antagonist properties for use in treating schizophrenia. According to this aspect and in some embodiments, such combination therapy includes the described therapeutic use or method of treatment including co-administration of the psychedelic drug and antipsychotic possessing 5-HT2A antagonist properties in a single composition, or in two different compositions administered in a sequence and/or within a period of seconds to days, or using a kit of parts with both components.

According to this aspect, and in some embodiments, the psychedelic drug is psilocybin, Dimethyltryptamine (DMT), 5-Methoxy-N,N-dimethyltryptamine (5-MeO-DMT), Mescaline, 3,4-methylenedioxymethamphetamine (MDMA) or Lysergic acid diethylamide (LSD) or a combination thereof and the antipsychotic possessing 5-HT2A antagonist properties is clozapine, olanzapine, quetiapine, risperidone, aripiprazole, asenapine, pizotifen, ziprasidone, chloropromazine, thioridazine, paloperidone, iloperidone, lurasidone, chlorprothixene, sertindole, lumateperone, zuclopenthixol, loxapine, amisulpiride, thiothixene, zotepine, cariprazine or a combination thereof.

In some aspects, the combination therapy comprises psilocybin and clozapine, wherein said clozapine is provided at a dosage of 100-400 mg as either a daily dosage or a bolus dosage. According to this aspect and in some embodiments the psilocybin is provided at a dosage of 1-5 mg as a daily dosage and in some aspects, the psilocybin is provided at a dosage of 2.5-25 mg as a bolus dosage.

In other aspects of the invention, including any of the methods of treatment and/or therapeutic uses as described herein, a combination therapy comprising a psychedelic drug and an NMDA receptor agonist is contemplated as is its use for treating a disease or disorder as herein described.

In some aspects, the NMDA receptor agonist is D-serine.

According to this aspect, and in some embodiments, the psychedelic drug is psilocybin, Dimethyltryptamine (DMT), 5-Methoxy-N,N-dimethyltryptamine (5-MeO-DMT), Mescaline, 3,4-methylenedioxymethamphetamine (MDMA) or Lysergic acid diethylamide (LSD) or a combination thereof.

In some aspects the NMDA receptor agonist is D-serine and the psychedelic drug is psilocybin, wherein the psilobycin is provided at a dosage of 1-5 mg as a daily dosage.

In other aspects, the psilocybin is provided at a dosage of 1-30 mg as a bolus dosage.

According to these aspects, the D-serine is provided at a dosage of about 1.5 grams-15 grams as a daily or bolus dosage administration.

In another aspect, the NMDA receptor agonist is at least a partial agonist. In some aspects, the NMDA receptor agonist is D-cycloserine.

According to this aspect, and in some embodiments, the psychedelic drug is psilocybin, Dimethyltryptamine (DMT), 5-Methoxy-N,N-dimethyltryptamine (5-MeO-DMT), Mescaline, 3,4-methylenedioxymethamphetamine (MDMA) or Lysergic acid diethylamide (LSD) or a combination thereof.

In some aspects the NMDA receptor agonist is D-cycloserine and the psychedelic drug is psilocybin, wherein the psilobycin is provided at a dosage of 1-5 mg as a daily dosage.

In other aspects, the psilocybin is provided at a dosage of 1-30 mg as a bolus dosage.

According to these aspects, the D-cycloserine is provided at a dosage of about 100 mg-1500 mg as a daily or bolus dosage administration.

In some aspects, reference to bolus administration includes administration of the indicated drug, from as frequently as once a week to once a month, and the skilled clinician will know how to adjust the frequency of administration depending on the clinical presentation of the subject in need.

In some aspects, reference to daily administration of the indicated compounds/drugs, is intended to include administration on a daily or essentially daily basis for from 1 to 4 weeks. In some aspects, reference to “daily administration” is also intended to include the possibility of alternate day dosing as well as 3 times, twice or once weekly administrations, as will be appreciated by the skilled artisan.

Administration of the compositions described herein to a patient, preferably a mammal, more preferably a human, may be carried out using known procedures, at dosages and for periods of time effective to treat a neurological disease or disorder or treat a disease or disorder that is affected by, associated with, or would benefit from the combination therapies described herein, in the patient. An effective amount of the therapeutic compound necessary to achieve a therapeutic effect may vary according to factors such as the state of the disease or disorder in the patient; the age, sex, and weight of the patient; and the ability of the therapeutic compound to treat a neurological disease or disorder or treat a disease or disorder that is affected by, associated with, or would benefit from the combination therapies as herein described, in the patient.

Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient. In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In other embodiments, the compositions are specifically formulated to promote delayed absorption of active ingredients as described herein in oral formulations, or in some embodiments, in dermally applied formulations, such as skin patches and the like. The skilled artisan will readily recognize the means to accomplish same, which include standard pharmaceutical preparations known in the art.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 pg to about 10,000 mg, about 20 pg to about 9,500 mg, about 40 pg to about 9,000 mg, about 75 pg to about 8,500 mg, about 150 pg to about 7,500 mg, about 200 pg to about 7,000 mg, about 350 pg to about 6,000 mg, about 500 pg to about 5,000 mg, about 750 pg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In various embodiments, the dose of a compound described herein is from about 1 mg and about 2,500 mg. In various embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in various embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms in a patient of a disease or disorder as described herein.

Formulations may be employed in admixtures with conventional excipients, pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like.

Routes of administration of any one of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, sachets, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

In some embodiments, it is particularly envisioned that when large dosages are administered, for example, D-SER and for example, in combination with PSIL, that a powder or solid form may be used, and in some embodiments, the active components will then be reconstituted into the water into which the powder is applied, for case of administration to the subject. It will be appreciated that additional flavorings, excipients may be included.

It should be understood that the formulations and compositions described herein are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch gly collate); or wetting agents (e.g., sodium lauryl sulphate). If desired, the tablets may be coated using suitable methods and coating materials such as OP ADR Y™ film coating systems available from Colorcon, West Point, Pa. (e.g., OP ADR Y™ OY Type, OYC Type, Organic Enteric OY-P Type, Aqueous Enteric OY-A Type, OY-PM Type and OP ADR Y™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodi acetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% a-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, pol oxamer 407, pol oxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. patents applications Ser. Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

In certain embodiments, the formulations described herein can be, but are not limited to, short-term, rapid-offset, as well as controlled, for example, sustained release, delayed release and pulsatile release formulations. The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body. Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

The term “therapeutically effective amount,” as used herein, refers to the amount of the indicated agent or agents, that, when administered to an individual is effective to at least partially treat a disorder, disease or condition from which the individual is suffering, or to at least partially ameliorate a symptom of such disorder, disease or condition. As is understood in the art, the therapeutically effective amount of a given compound will depend at least in part upon, the mode of administration, any carrier or vehicle (e.g., solution, emulsion, etc.) employed, the specific disorder or condition, and the specific individual to whom the compound is to be administered (age, weight, condition, sex, etc.).

The dosage requirements need to achieve the “therapeutically effective amount” vary with the particular compositions employed, the route of administration, the severity of the symptoms presented and the particular subject being treated. Based on the results obtained in standard pharmacological test procedures, projected daily dosages of active compounds can be determined as is understood in the art.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

All publications, patents, and patent applications mentioned herein are hereby incorporated by reference in their entirety as if each individual publication or patent was specifically and individually indicated to be incorporated by reference. In case of a conflict between the specification and an incorporated reference, the specification shall control. Where number ranges are given in this document, endpoints are included within the range. Furthermore, it is to be understood that unless otherwise indicated or otherwise evident from the context and understanding of one of ordinary skill in the art, values that are expressed as ranges can assume any specific value or subrange within the stated ranges, optionally including or excluding either or both endpoints, in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

In the claims articles such as “a,”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

Unless the context indicates otherwise, it is specifically intended that the various features described herein can be used in any combination.

In some aspects, unless specified otherwise, the terms “reduce,” “decrease,” “lessen” and similar terms mean a decrease of at least about 10%, about 15%, about 20%, about 25%, about 35%, about 50%, about 75%, about 80%, about 85%, about 90%, about 95%, about 97%, or more.

Similarly, in other aspects, unless specified otherwise, the terms “improve,” “increase,” “enhance,” and similar terms indicate an increase of at least about 10%, about 15%, about 20%, about 25%, about 50%, about 75%, about 100%, about 150%, about 200%, about 300%, about 400%, about 500%, or more.

In some aspects, unless specified otherwise, the reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

All disease and disorders listed herein are defined as described in the Diagnostic and Statistical Manual of Mental Disorders (DSM-5), published by the American Psychiatric Association, or in International Classification of Diseases (ICD), published by the World Health Organization.

As used herein the term “subject” and “patient” are used interchangeably.

As used herein, “treating” and like terms refer to reducing the severity and/or frequency of one or more symptoms, eliminating one or more symptoms and/or the underlying cause of said symptoms, reducing the frequency or likelihood of one or more symptoms and/or their underlying cause, delaying, preventing and/or slowing the progression of diseases and/or disorders and improving or remediating damage caused, directly or indirectly, by the diseases and/or disorders.

It will be appreciated, that in reference to any method of this invention or combination, or treatment regimen or composition or kit comprising therapeutic agents as herein described, that the invention specifically contemplates that the compound/drug/agent is provided in a therapeutically effective dose.

As used herein, the term “therapeutically-effective dose” means a dose sufficient to achieve the intended therapeutic purpose, such as, to alleviate a sign or symptom of a disease or disorder in a subject.

The following examples describe certain embodiments of the invention and should not be construed as limiting the scope of what is encompassed by the invention in any way.

### EXAMPLES

### Example 1

### Clozapine and D-Serine Reduce or Abrogate Psychedelic Trip Effects Induced by Psilocybin

The head twitch response (HTR) induced by serotonergic psychedelic agents such as psilocybin (PSIL) in rodents, is a characteristic behavioral effect mediated by cortical 5-HT2A receptors that is highly correlated with the psychedelic trip induced by these agents in humans.

HTR induced by PSIL in C57Bl/6j mice has been extensively characterized.

In order to determine whether the negative effects such as HTR could be blocked in schizophrenic patients administered psilocybin (PSIL) (which ordinarily would produce “trip-induced effects”) upon further receiving clozapine (CLZ), the effect of concurrent CLZ treatment in blocking the HTR-inducing effect of PSIL in mice was examined.

Toward this end, male ICR mice (aged 8 weeks) were implanted with mini car-magnets and were administered the following treatments intraperitoneally (i.p.):
1. CLZ at a dosage of 1 or 2 mg/kg weight;
2. PSIL at a dosage of 4.4 mg/kg weight;
3. CLZ at a dosage of 1 mg/kg weight and PSIL at a dosage of 4.4 mg/kg weight;
4. CLZ at a dosage of 2 mg/kg weight and PSIL at a dosage of 4.4 mg/kg weight; and
5. Vehicle (Saline) controls.

CLZ was administered 30 minutes before PSIL. All injection volumes were identical. HTR was assessed in up to 6 mice simultaneously over a 20-minute period using a purpose-built magnetometer and custom software. Experiments were approved by the Authority for Biological and Biomedical Models, Hebrew University

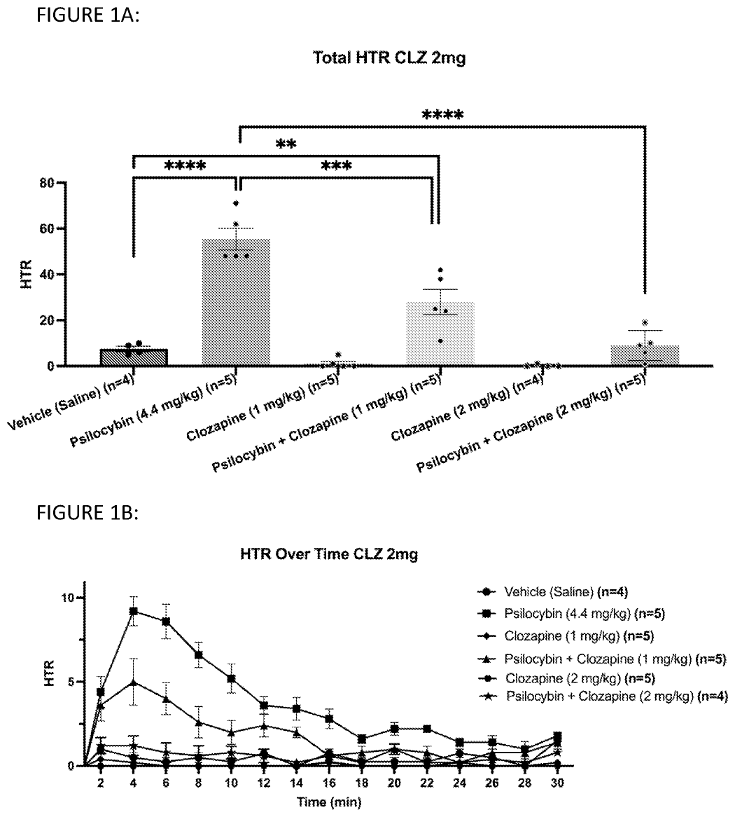

FIG. 1A shows the total HTR in each of the 5 treatment groups over a 20-minute period (mean+standard error). PSIL induced a significant increase in total HTR over the observation period in comparison to controls. CLZ treatment alone, whether at a dosage of 1 or 2 mg/kg weight did not affect HTR. Administration of CLZ at a dosage of 1 mg/kg prior to PSIL significantly reduced PSIL-mediated increased HTR and administration of CLZ at a dosage of 2 mg/kg weight abrogated PSIL-mediated HTR entirely, resulting in control levels.

To further extend these studies to determine the effects over time, HTR was assessed and plotted for up to 30 minutes to confirm the rapid abrogation of PSIL-mediated increased HTR in animals administered CLZ (FIG. 1B).

Thus administration of CLZ at a dosage equivalent to that administered clinically to humans blocked PSIL-mediated HTR in mice. These results support that CLZ, when co-administered with PSIL in humans, may reduce or abrogate the psychedelic trip, since the HTR rodent model has been shown to be highly extrapolatable to that of psychedelic trip in humans.

These studies indicate that CLZ will positively affect the PSIL-induced negative effect of psychedelic trip.

PSIL administration has been shown to provide positive neuroplastic effects of the drug in murine models. These positive effects may not be 5-HT2A receptor mediated, since the effects were observed in conditions where there was concurrent administration of a 5-HT2A receptor blocker

Administering the atypical antipsychotic CLZ, which has 5-HT2A antagonist properties to patients that receive PSIL is, in view of the above results, expected to block the trip-inducing effects of PSIL (which could exacerbate psychosis) but not in any way interfere with the 5-HT2A receptor independent positive effects of PSIL treatment, whereby negative symptoms of the anti-psychotic are ameliorated or abrogated, while positive effects are enhanced.

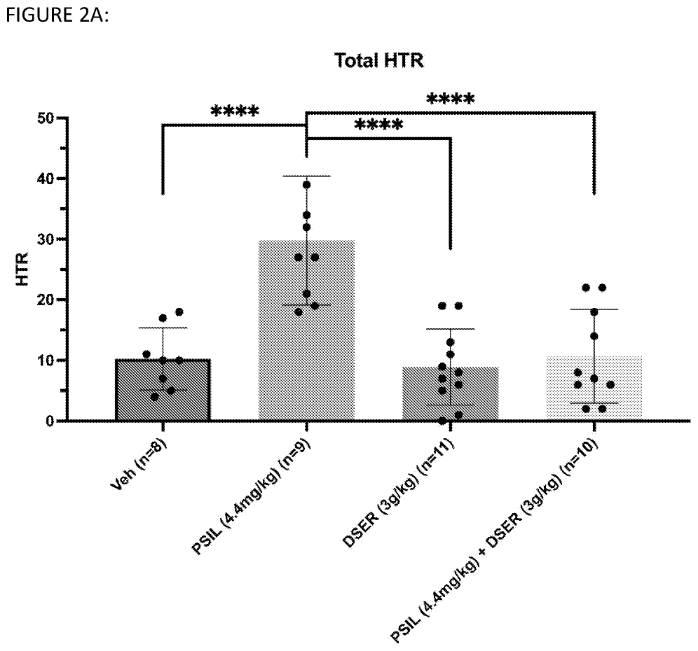

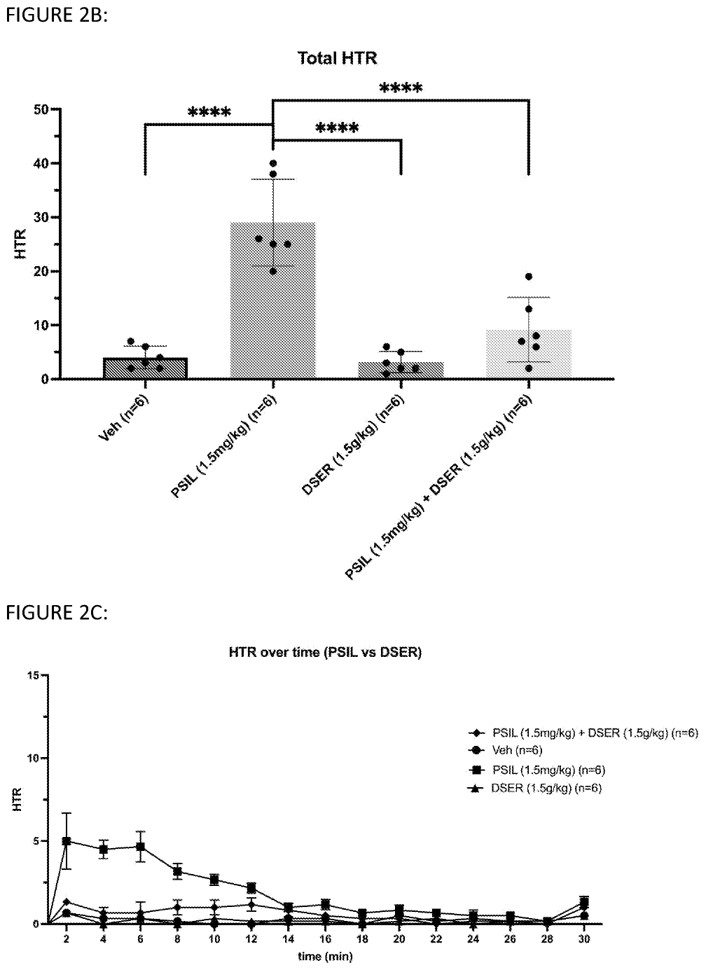

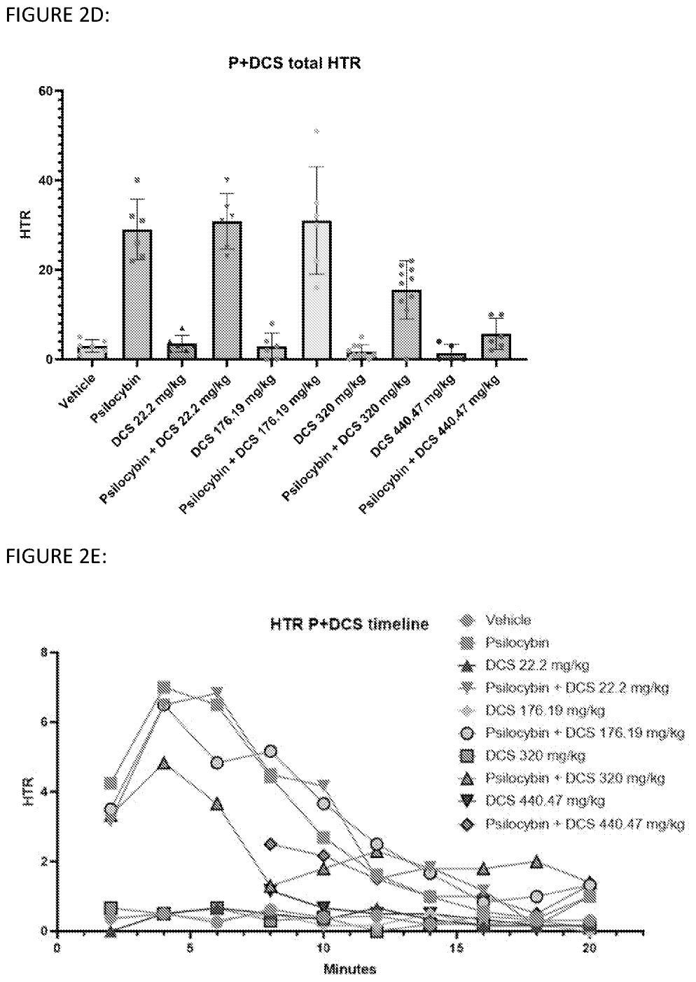

To further extend these studies, it was of interest to determine the HTR effects induced by Psilocybin when mice were pre-administered D-Serine 3000 mg/kg, as opposed to CLZ (FIG. 2A), or D-Serine 1500 mg/kg, as opposed to CLZ (FIG. 2B). Similar to CLZ, D-Serine significantly reduced PSIL-mediated increased HTR. While DSR alone did not affect HTR, mice co-administered DSR and psilocybin promoted complete HTR blockade (p=0.0022-DSER 3000+psilocybin vs. psilocybin). The effect was also consistent over time, even with the lower D-serine dose (FIG. 2C)

When assessing the effects on HTR induced by PSIL in mice treated with D-cycloserine (DCS), DCS alone, similar to DSR, did not affect HTR. The combination of psilocybin and DCS, however, attenuated HTR and induced a significantly lower number of head twitches compared to psilocybin alone (p=0.0001) (FIG. 2D), when doses of DCS above 320 mg/kg were utilized. Similar to what was seen in FIG. 1B, when assessing HTR plotted for up to 30 minutes, the rapid abrogation of PSIL-mediated increased HTR in animals administered DCS at doses of over 320 mg/kg (FIG. 2E).

### Example 2

### Effect of Combination Therapies on Hyperactivity Induced by the NMDA Receptor Antagonist, MK-801

Hyperactivity induced in rodents by the NMDA receptor antagonist, MK-801, is a key rodent pharmacological model of acute psychotic features in humans.

Drugs which induce psychosis in humans enhance MK-801 induced hyperactivity in rodents while antipsychotic drugs reduce or block the effect.

In one aspect of this invention, combination therapies/compositions/methods are provided for the treatment for chronic schizophrenia, through the co- or staggered administration of a psychedelic agent, such as PSIL in combination with an atypical antipsychotic drug, CLZ.

According to this aspect, the possibility that PSIL might induce psychosis in these patients would be expected to be blocked by CLZ, inter alia, based on the results of Example 1. It is further an aspect of this invention, that PSIL will not impede the antipsychotic effect of CLZ.

In order to determine the interactive effects of PSIL and CLZ, a murine model was used where MK-801 induced hyperactivity, was assessed, which serves as a surrogate system for psychotic symptoms in schizophrenia patients.

Toward this end PSIL and CLZ alone and in combination were evaluated for their effects on MK-801 induced hyperactivity in C57Bl/6j mice.

ICR mice were administered MK-801 intra-peritoneally, at a dose of 0.1 mg/kg immediately before they were placed in an open field for one hour. 30 minutes prior to administration of MK-801, mice were pretreated as follows:
1. PSIL at a dosage of 4.4 mg/kg;
2. CLZ at a dosage of 1.0 mg/kg;
3. PSIL at a dosage of 4.4 mg/kg and CLZ at a dosage of 1.0 mg/kg;
4. D-Serine (DSR) at a dosage of 3000 mg/kg;
5. D-cycloserine (DCS) at a dosage of 320 mg/kg;
6. PSIL at a dosage of 4.4 mg/kg and DSR at a dosage of 3000 mg/kg;
7. PSIL at a dosage of 4.4 mg/kg and DCS at a dosage of 320 mg/kg; and
8. Saline (Vehicle) Controls (n=8).

All injection volumes were identical and provided intraperitoneally.

Activity in the open field was monitored by an overhead camera and was recorded by a Noldus Ethovision apparatus. Experiments were approved by the Authority for Biological and Biomedical Models, Hebrew University. Distance moved/travelled in the open field, center duration and center frequency were evaluated in the open field arena.

FIG. 3A shows distance travelled in the open field, where MK-801 (referred to as “MK” in FIG. 3A) treatment produced marked movement, reflecting MK-801 induced hyperactivity.

It is noteworthy that pretreatment with PSIL did not increase MK-801 induced hyperactivity.

Pretreatment with CLZ significantly reduced MK-801 induced hyperactivity and its co-administration with PSIL resulted in comparable effects to treatment with CLZ alone, i.e. PSIL did not abrogate CLZ's ability to significantly reduce MK-801 induced hyperactivity.

These findings further in the context of the results of Example 1, help deduce certain unique aspects of this invention.

As a first matter, the administration of PSIL at a dose that induces significant HTR does not enhance MK-801 induced hyperactivity.

Furthermore, CLZ concurrent treatment with PSIL does not result in an overcoming of CLZ blockade of MK-801 induced hyperactivity.

It was therefore of interest to determine whether mice administered D-Serine or D-cycloserine, affected the MK-801 induced hyperactivity.

In the acute MK-801-induced hyperlocomotion test, a significant difference (p=0.02) was observed in the distance travelled between mice administered saline prior to MK-801 injection and mice administered DSR and psilocybin (FIG. 3B). The DSR and psilocybin treated group moved a significantly shorter distance, indicative or lower potential of the DSR and psilocybin treatment combination to induce or exacerbate positive psychotic symptoms (FIG. 3C). Similar results were obtained when co-administration of DCS and psilocybin was assessed, where the hyperactivity induced by MK-801 is significantly reduced (p=0.008) (FIG. 3D-3E), yet the DCS and psilocybin treated group, as well, moved a significantly shorter distance, indicative or lower potential of the DCS and psilocybin treatment combination to induce or exacerbate positive psychotic symptoms.

results Together these support the proposed combination therapy/compositions/methods of this invention, for concurrent chronic schizophrenia treatment with PSIL and CLZ, DSR or DCS, without exacerbating acute psychotic symptoms in these individuals.

### Example 3

### Effect of Combined PSIL and CLZ Treatment in Promoting Neuroplasticity

In order to further establish the positive effects of the treatment regimens and compositions of this invention, to ascertain whether same influences or otherwise positively impacts neuroplasticity, an in vivo model was evaluated.

Specifically, ICR mice were administered a single intra-peritoneal injection of psilocybin at a dosage of 4.4 mg/kg, D-Serine at a dosage of 3 g/kg, a combined psilocybin dosage of 4.4 mg/kg+D-Serine dosage of 3 g/kg and compared to control animals (administered a saline vehicle). 7 days post-administration, animals were harvested, and individual brain regions were dissected, homogenized and processed for Western Blot analysis, probing for GAP43, Synaptophysin and SV2A protein expression, using commercially available reagents (Abcam).

Animals injected with PSIL or D-SER or the combination of the two were specifically probed for changes in synaptic protein expression, for proteins that are pivotally involved in neuroplasticity of the brain, focusing on 4 key regions: the brain frontal cortex, hippocampus, amygdala and striatum.

When assessing the synaptic protein, GAP43, expression levels, it was found that GAP43 protein levels were significantly increased by D-Serine administration, in the frontal cortex and amygdala of subjects administered D-SER alone. When combining D-SER and PSIL, GAP43 protein expression increased significantly in the hippocampus and amygdala (FIG. 4).

D-Serine singular administration resulted in the increased synaptic protein expression of synaptophysin, in the frontal cortex and SV2A expression in the hippocampus, whereas the combined administration of DSER and PSIL resulted in significant increased SV2A expression in the hippocampus (FIG. 5).

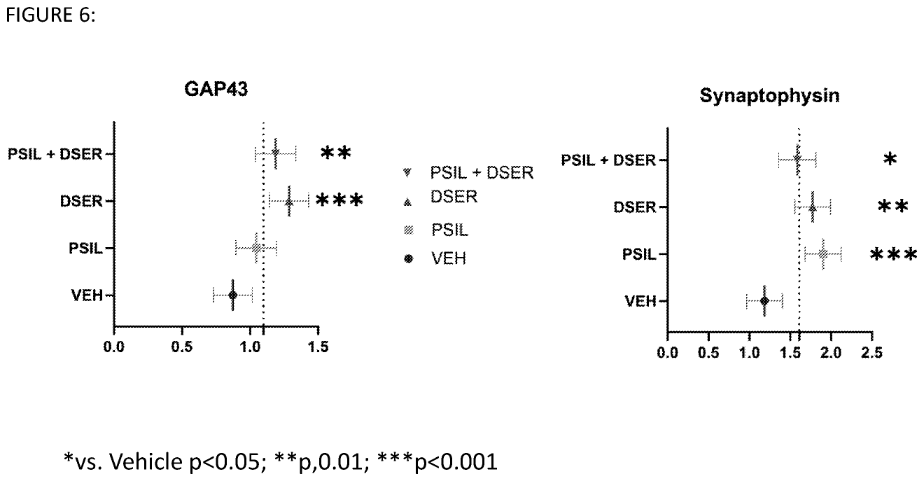

In order to comparatively assess all the effects of individual DSER or PSIL and combined DSER and PSIL administration on each brain region, a nested analysis of variance in which the level each of the synaptic proteins was compared to controls was assessed across the four brain regions frontal cortex, hippocampus, amygdala and striatum (FIG. 6). The results for GAP43 show that D-Serine and D-Serine+Psilocybin increase GAP43 levels across all 4 brain regions, while psilocybin alone does not. Synaptophysin expression is positively impacted by all three treatments, DSER and PSIL individually and combined, in increasing levels of the synaptic protein across all 4 brain regions.

Taken together, these results show a clear effect of D-Serine, alone and in combination with psilocybin to enhance synaptic protein levels, while the effect of PSIL alone is far less pronounced.

Emerging from these results is clear support for the combined treatment with psilocybin and D-Serine enhancing neuroplasticity.

### Example 4

### Effect of a Psychedelic Compound in Combination with NMDA Modulators in the Treatment of Schizophrenia

In addition to the hypothesized positive effect of atypical anti-psychotics such as CLZ, with psychedelic agents, it is of interest to evaluate the effect of psychedelic agents in combination with NMDA Modulators,

In a first study treatment effects with different combinations of psilocybin are evaluated in known disease models of schizophrenia: (i) neonatal administration of the NMDA glutamatergic receptor antagonist, MK-801 that models positive, negative and cognitive features; and, (ii) acute injection of MK-801 to adult mice that specifically models positive features.

Thus, psilocybin (PSIL at acute psychedelic dose of 4.4 mg/kg), chronically (more than 14 days) and/or subchronically (several times but less than 14 days) with sub-psychedelic dose determined in a prior dose response experiment is used as the psychedelic agent. The effects of PSIL in combination with the atypical antipsychotic drug, clozapine (CLZ at 10 mg/kg) or the NMDA modulator, D-serine (at 1000 mg/kg) are further evaluated as described herein.

In the first experiment, administration of PSIL given for more than 14 days i.p. at a sub-psychedelic dose (determined in a prior dose response experiment) and/or subchronically (several times but less than 14 days), alone or in conjunction with CLZ or D-serine, and its beneficial effects on positive, negative and cognitive schizophrenia like phenotypes induced by the neonatal MK-801 disease model (administration of MK-801 at 0.5 mg/kg during post-natal days [PNDs] 6-13), will be examined.

It is expected that both chronic (21 days) and/or subchronic (several times but less than 14 days), sub-psychedelic doses of PSIL and an acute psychedelic dose of PSIL will have robust effects on neonatal MK-801 induced schizophrenia like phenotypes.

Concurrent treatment of PSIL and CLZ will prevent positive-like features of schizophrenia on the acute, adult MK-801 model, while concurrent treatment with CLZ or D-serine enhances the therapeutic effects of PSIL as observed in the neonatal MK-801 schizophrenia model.

It is further expected that sub-psychedelic doses of PSIL in conjunction with the atypical antipsychotic drug, clozapine (CLZ) or the NMDA receptor agonist, D-serine, will have a more beneficial effect on positive, negative and cognitive schizophrenia like phenotypes induced by the MK-801 neonatal schizophrenia model as compared to PSIL alone.

In a further study, neonatal administration of MK-801 will again be used as the schizophrenia model. PSIL will be administered at a psychedelic dose to mice pretreated chronically (21 days) with CLZ or D-serine and the behavioral and cognitive effects will be determined. On PND 70, treatment will commence with clozapine or D-serine. Psilocybin at a psychedelic dose will be administered on PND-80 prior to behavioral tests ending on PND 90.

It is expected that the psychedelic dose of PSIL will have a robust beneficial effect on schizophrenia-like phenotypes in the neonatal MK-801 model of schizophrenia used in this study; that a psychedelic dose of PSIL given in conjunction with CLZ will not induce exacerbation of positive-like symptoms and that a psychedelic dose of PSIL in conjunction with the atypical antipsychotic drug, clozapine (CLZ) or the NMDA receptor agonist, D-serine, will have a more beneficial effect on positive, negative and cognitive schizophrenia like phenotypes induced by the MK-801 neonatal schizophrenia model than PSIL alone.

In both this and the previous study, social tests are conducted, via the addition of social stimulus animals. A social stimulus animal is not pharmacologically treated. Its sole purpose is to interact socially, in one of two of the social tests: the three chambers sociability and social novelty test (3CHAMB) and the social interaction in pairs test (SIIP), with the pharmacologically treated test animals. In the 3CHAMB test, the treated test animal is interacting with two social stimulus animals whose sole purpose is to interact with the test animal as part of the test protocol. In the SIIP test, similarly, each pharmacologically test animal has to interact with one social stimulus animal, as part of this behavioral test protocol. Therefore, the social stimulus animals are counted apart from the treated test animals. They are not treated and their purpose is to serve a social challenge to the treated test animals.

In both this and the previous study, a_Y-maze evaluation will be conducted, as well, whereby animals are introduced inside the maze apparatus and left to roam it for 6 minutes. Complete triplets (animal is entering the three arms one after the other, such as ABC or BCA) direct visits (animal entering the same arm in exited) and indirect visits are monitored, which are performed at PND 85.

In yet a further study, the disease model includes an acute MK-801 injection to adult mice, to determine whether sub-psychedelic doses of PSIL given alone or in conjunction with sub-chronic CLZ or D-serine worsens positive symptoms, via use of the acute adult MK801 model of schizophrenia. On PND 70 animals will begin to receive psilocybin for 10 days, at a sub psychedelic dose, alone or in combination with clozapine or D-serine. On PND 80, MK-801 injection will be performed followed by behavioral tests (open field activity immediately, pre pulse inhibition the day after).

In yet a further study, the disease model includes again the acute MK-801 injection to adult mice and evaluation as to whether the treatment regimen of a psychedelic dose of PSIL given in conjunction with sub-chronic CLZ or D-serine worsens positive symptoms, which are effectively modeled by acute MK-801, is determined. On PND 70, treatment will commence with clozapine or D-serine. Psilocybin at a psychedelic dose will be administered on day 80 prior to MK-801 and the same behavioral tests as mentioned immediately hereinabove, is performed.

As noted, Open field tests (OFT) will be conducted, performed (i) at baseline before starting treatment; (ii) during the behavioral battery on PND 80 in all these studies, where velocity and time spent in the arena center or circumference is assessed.

For these studies it is noted that the dosages of the various agents are as follows, Psilocybin 4.4 mg/kg+MK-801 0.1 mg/kg, D-serine 1000 mg/kg.

In order to test whether PSI has an exacerbating effect on psychosis, Pre-pulse inhibition (PPI) studies, a model for positive symptoms, are used. Animals are placed inside a well-ventilated restrainer inside a specialized chamber designed to monitor animal twitch response to various sound stimuli. In the beginning, animals are exposed to 68 dB white background noise. 10 pulses each 120 dB for 40 msec starts the protocol, after which a combination of such pulses preceded with pre-pulse sound stimuli 4, 8 and 16 dB above the background noise. Animal is recorded, and the testing is performed (i) at baseline before starting treatment; (ii) during the behavioral battery on PND 83 in the first two studies or on PND 81 a day after acute MK-801 injections in the latter two studies.

In this manner, we are able to assess the improvement of symptoms and the disease course, in particular in terms of the negative symptoms of same, via the use of the PSIL+D-Serine combination and/or PSIL+CLZ in treating schizophrenia, as evidenced in these model systems.

### Example 5

### Effect of Combined PSIL and CLZ Treatment in Chronic Schizophrenia and Other Psychiatric Disorders in Clinical Trials

In addition to the proposed efficacy regarding treatment regimens/compositions/combinations for chronic schizophrenia, the combination therapy/compositions/methods of this invention are expected to also be useful in treating other psychiatric disorders such as treatment resistant depression or PTSD.

In some aspects, the invention specifically provides combination therapy/compositions/methods for treating depression and/or PTSD or related psychiatric disorders that do not respond to conventional therapy.

In these aspects, the combination therapy/compositions/methods of this invention may include the administration of a tryptaminergic psychedelic drug, including, but not limited to psilocybin, at a dosage of 0.28-0.35 mg/kg, or in some aspects, another psychedelic drug that acts via the 5-HT2A receptor may be used.

According to this aspect and in some embodiments, the psychedelic drug will be provided with or in a proximate staggered manner to patients receiving treatment with clozapine for at least 2 weeks at a dose of 2.8-5.6 mg/kg.

According to some aspect, the psychedelic drug, for example, psilocybin is administered once weekly, for 4 weeks concurrently with clozapine treatment.

According to some aspects, the psychedelic drug, for example, psilocybin is provided at a lower dosage of 0.25-0.30 mg/kg weekly for 4 weeks.

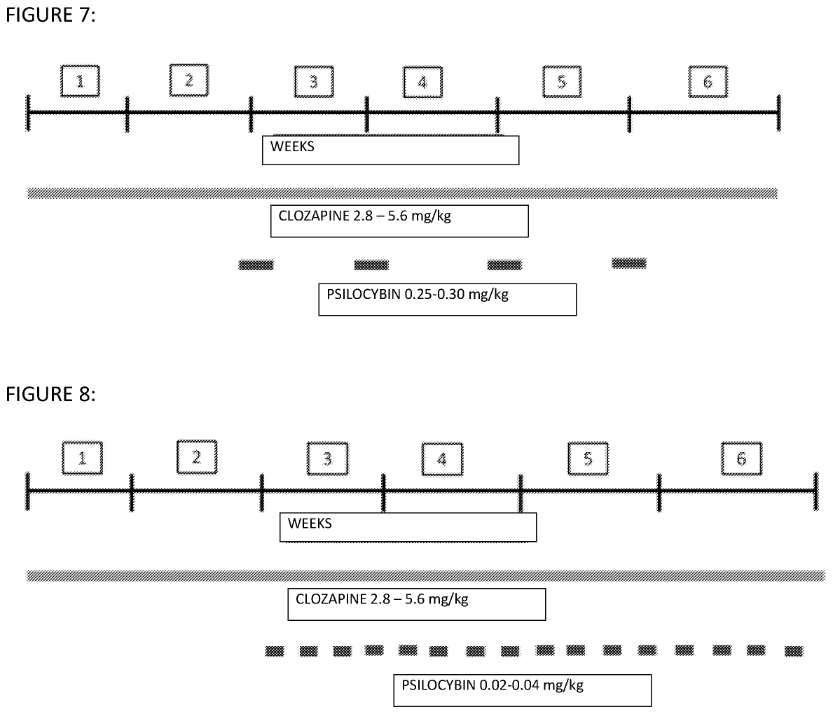

It is expected that due to its 5-HT2A receptor blocking properties, clozapine will eliminate the trip-inducing effects of psilocybin while allowing the neuroplastic effects to be induced (FIG. 7.)

According to some aspects, the psychedelic agent such as psilocybin is administered every second day for 4 weeks to patients with chronic schizophrenia or another psychiatric disorder such as treatment resistant depression or PTSD that does not respond to conventional therapy at a dose that is insufficient to induce psychedelic effects (0.02-0.04 mg/kg) (or another psychedelic drug that acts via the 5-HT2A receptor, administered at a sub-psychedelic dose according to the same regimen).

According to some aspects, the psilocybin is administered concurrently with clozapine at a daily dose of 2.8-5.6 mg/kg, initiated at least two weeks prior to the inception of treatment with psilocybin (FIG. 8).

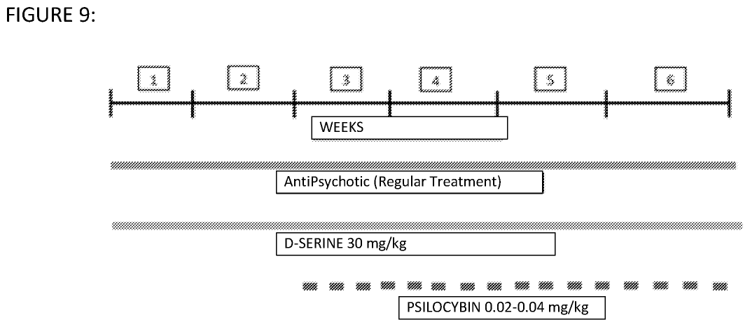

According to some aspects, the invention contemplates a further treatment strategy in which patients with chronic schizophrenia or another psychiatric disorder such as treatment resistant depression or PTSD that does not respond to conventional therapy (whether treated on an ongoing basis with an antipsychotic drug other than clozapine or not) are concurrently administered D-serine at a dose of at least 30 mg/kg and psilocybin at a dose of 0.02-0.04 mg/kg (or a sub-psychedelic dose of another psychedelic drug that acts via the 5-HT2A receptor). D-serine is administered daily for two weeks prior to inception of psilocybin (or another psychedelic drug at a sub-psychedelic dose) which is then administered at a dose of 0.02-0.04 mg/kg every second day for 4 weeks concurrently with D-serine. We hypothesize that psilocybin and D-serine will have additive neuroplasticity-promoting effects while D-serine will have a protective role versus possible positive symptom-like effects of psilocybin (FIG. 9).

According to this aspect, analogous treatment strategies to those implemented in schizophrenia, may be implemented in treatment resistant depression and in chronic PTSD that is non-responsive to therapy. Still further, according to this aspect, D-cycloserine may be substituted for D-serine in all the above treatment strategies.

According to this aspect, it is anticipated that one or more of these novel treatment strategies will bring about improvement in the negative symptoms of schizophrenia without exacerbating the positive symptoms of the illness and also in treatment resistant depression and PTSD that do not respond to conventional therapy.

In another aspect, the combinations/compositions/treatment/uses of this invention contemplate incorporation of same with a regimen involving cognitive behavioral therapy (CBT). In some embodiments, prior to a CBT session, or prior to one out of 2 or one out of 3, etc. CBT sessions, the invention contemplates administration of the combinations/compositions as herein described, such as, for example, providing, prior to each session (or every other 2 or 3 sessions) a compound including full dose PSIL and CLZ, in some embodiments, or PSIL and an NMDAR agonist, such as, for example, D-cycloserine or D-serine. In some aspects, the combinations are expected to outperform the use of either compound, individually.

Both DSR and DCS are expected to limit the acute hallucinogenic effects of psilocybin, as reflected by the influence of these compounds on the head twitch response demonstrated hereinabove.

This supports the feasibility of co-administration of these compounds to patients at risk for induction or exacerbation of psychosis.

Moreover, the combination of DSR and psilocybin and of DCS and psilocybin significantly reduced MK-801-induced hyperlocomotion, supporting their therapeutic effect on positive symptoms of schizophrenia, while Psilocybin alone did not exacerbate MK-801-induced hyperactivity.

These Examples thus demonstrate that the claimed compositions/combinations/methods/kits/uses of the invention relying on these unique combination therapies can significantly improve the treatment of refractory psychiatric disorders by addressing NMDA receptor-mediated effects while leveraging neuroplastic properties of psilocybin.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

It will be understood by those skilled in the art that various changes in form and details may be made therein without departing from the spirit and scope of the invention as set forth in the appended claims. Those skilled in the art will recognize, or be able to ascertain using no more than routine experimentation, many equivalents to the specific embodiments of the invention described herein. Such equivalents are intended to be encompassed in the scope of the claims.

In one embodiment of this invention, “about” refers to a quality wherein the means to satisfy a specific need is met, e.g., the size may be largely but not wholly that which is specified but it meets the specific need of cartilage repair at a site of cartilage repair. In one embodiment, “about” refers to being closely or approximate to, but not exactly. A small margin of error is present. This margin of error would not exceed plus or minus the same integer value. For instance, about 0.1 micrometers would mean no lower than 0 but no higher than 0.2. In some embodiments, the term “about” with regard to a reference value encompasses a deviation from the amount by no more than 5%, no more than 10% or no more than 20% either above or below the indicated value.

In the claims articles such as “a”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

1-151. (canceled)

152. A combination therapy comprising a psychedelic drug and an NMDA receptor agonist.

153. The combination therapy of claim 152, wherein said NMDA receptor agonist is at least a partial agonist.

154. The combination therapy of claim 152, wherein said NMDA receptor agonist is D-serine.

155. The combination therapy of claim 152, wherein said NMDA receptor agonist is D-cycloserine.

156. The combination therapy of claim 152, wherein said psychedelic drug is psilocybin, dimethyltryptamine (DMT), 5-methoxy-N,N-dimethyltryptamine (5-MeO-DMT), mescaline, 3,4-methylenedioxymethamphetamine (MDMA), lysergic acid diethylamide (LSD), or a combination thereof.

157. The combination therapy of claim 156, wherein said NMDA receptor agonist is D-serine.

158. The combination therapy of claim 157, wherein said psilocybin is provided at a dosage of 1-5 mg as a daily dosage, or wherein said psilocybin is provided at a dosage of 1-30 mg as a bolus dosage, and wherein said D-serine is provided at a dosage of 1.5-15 g as a daily or bolus dosage administration.

159. The combination therapy of claim 156, wherein said NMDA receptor agonist is D-cycloserine.

160. The combination therapy of claim 159, wherein said psilocybin is provided at a dosage of 1-5 mg as a daily dosage, or wherein said psilocybin is provided at a dosage of 1-30 mg as a bolus dosage, and wherein said D-cycloserine is provided at a dosage of 100-1500 mg as a daily or bolus dosage administration.

161. The combination therapy of claim 152, further comprising an antipsychotic possessing 5-HT2A antagonist properties, wherein said antipsychotic possessing 5-HT2A antagonist properties is clozapine, olanzapine, quetiapine, risperidone, aripiprazole, asenapine, pizotifen, ziprasidone, chloropromazine, thioridazine, paloperidone, iloperidone, lurasidone, chlorprothixene, sertindole, lumateperone, zuclopenthixol, loxapine, amisulpiride, thiothixene, zotepine, cariprazine, or a combination thereof.

162. The combination therapy of claim 161, wherein said antipsychotic possessing 5-HT2A antagonist properties is clozapine, wherein said clozapine is provided at a dosage of 100-400 mg as either a daily dosage or a bolus dosage.

163. A kit comprising the combination therapy of claim 152.

164. A composition comprising the combination therapy of claim 152.

165. A method of treating, reducing the incidence of, reducing the severity of or improving a pathology of a subject with a neuropsychiatric disease or disorder, neurologic disease or disorder, neurocognitive disease or disorder, or motor disease or disorder, the method comprising administering to said subject the combination therapy of claim 152.

166. The method of claim 165, wherein said disease or disorder is schizophrenia, depression, post-traumatic stress disorder, obsessive-compulsive disorder (OCD), cerebral palsy, muscular dystrophy, spina bifida, spinal muscle atrophy (SMA), Parkinson's disease, epilepsy, amyotrophic lateral sclerosis (ALS), Ataxia, attention-deficit hyperactivity disorder, generalized anxiety disorder, panic disorder, cervical dystonia, general dystonia, chorea, functional movement disorder, Huntington's disease, multiple system atrophy, myoclonus, chronic pain, inflammation, Parkinsonism, Alzheimer's disease, sleep-wake disorders, Stroke, repeated head trauma, progressive supranuclear palsy, restless legs syndrome, tardive dyskinesia, Tourette syndrome, tremor, Wilson's disease, or an autism spectrum disorder (ASD), or a symptom thereof.

167. A method of treating, reducing the incidence of, reducing the severity of or improving a pathology of a subject with schizophrenia, the method comprising administering to said subject a combination of a psychedelic drug and an NMDA receptor agonist, wherein said NMDA receptor agonist is at least a partial agonist, wherein said NMDA receptor agonist is bitopertine, or wherein said NMDA receptor agonist is D-serine, or wherein said NMDA receptor agonist is D-cycloserine.

168. The method of claim 167, wherein said D-serine is deuterated D-serine.

169. The method of claim 167, wherein said psychedelic drug is psilocybin, wherein said psilocybin is provided at a dosage of 1-3 mg as a daily dosage, or wherein said psilocybin is provided at a dosage of 1-5 mg as a bolus dosage, and wherein said D-serine is provided at a dosage of 1.5-15 g as a daily or bolus dosage administration.

170. The method of claim 167, wherein said psilocybin is provided at a dosage of 1-5 mg as a daily dosage, or wherein said psilocybin is provided at a dosage of 1-30 mg as a bolus dosage, and wherein said D-cycloserine is provided at a dosage of 100-1500 mg as a daily or bolus dosage administration.

171. The method of claim 167, wherein said treating, reducing the incidence of, reducing the severity of or improving a pathology of a subject with schizophrenia comprises reducing or abrogating negative symptoms of schizophrenia.
